(19) United States
(12) Patent Application Publication
(10) Pub. No.: US 2010/0166726 A1
(43) Pub. Date: Jul. 1, 2010

(54) COMPOSITIONS FOR IMPROVING CELLULAR UPTAKE OF A CHEMOTHERAPEUTIC AGENT IN A CELL EXHIBITING MUCIN DEREGULATION

(75) Inventor: Robert B. Campbell, Quincy, MA (US)

Correspondence Address: WILMERHALE/BOSTON, 60 STATE STREET, BOSTON, MA, 02109, US

(73) Assignee: NORTHEASTERN UNIVERSITY, Boston, MA (US)

(21) Appl. No.: 12/530,339

(22) PCT Filed: Mar. 7, 2008
(86) PCT No.: PCT/US08/03020

§ 371 Date: Mar. 4, 2010

Related U.S. Application Data
(60) Provisional application No. 60/905,564, filed on Mar. 7, 2007.

(51) Int. Cl. A61K 38/43; A61K 31/505; C12N 5/00

(52) U.S. Cl. 424/94.5; 514/718; 514/274; 435/375

(57) ABSTRACT

Methods and compositions that increase the cellular uptake of a chemotherapeutic agent are provided. Also provided are methods of increasing access to the cell surface by antibodies and ligands. The methods are useful in treating any mucinous carcinoma characterized by an increased expression and/or secretion of mucins. In addition, these methods provide an improvement in existing methods of chemotherapy.

### CROSS REFERENCE TO RELATED APPLICATIONS

This application claims the benefit of U.S. Provisional Application No. 60/905,564, filed Mar. 7, 2007, the contents of which are incorporated herein by reference in its entirety.

### FIELD OF THE INVENTION

This application relates to the field of mucin biology. More specifically, this application relates generally to methods and compositions for improving the cellular uptake of a therapeutic agent in a subject in need thereof.

### BACKGROUND

Glycosylation is one of the major post-translational modifications found in greater than 50% of all proteins. There are three major types of protein glycosylation: O-glycosylation, N-glycosylation, and phosphatidylinositol-glycosylation. O-glycosylated proteins, themselves, can be divided into five different groups of which the mucin-type glycoproteins represent a major group. This group includes glycans with α-N-acetylgalactosamine (GalNAc) linked to serine or threonine side chains of proteins such as mucin.

Mucins are high molecular weight glycoproteins having oligosaccharides attached to a protein backbone core by O-glycosidic linkages and are approximately 50% to 80% carbohydrates in terms of total molecular mass. Mucins are synthesized as rod-shape apomucin cores that are post-translationally modified by significant glycosylation. The amino- and carboxy-terminal regions are very lightly glycosylated, but rich in cysteines that are likely involved in establishing disulfide linkages within and among mucin monomers. Mucins have a large central region formed of multiple tandem repeats of 10 to 80 residue sequences in which up to half of the amino acids are serine or threonine. This area becomes saturated with hundreds of O-linked oligosaccharides. N-linked oligosaccharides are also found on mucins, but much less abundantly.

Several human mucin genes have been cloned including, MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, and MUC19. The major secreted airway mucins are MUC5AC and MUC5B; whereas, MUC2 is secreted mostly in the intestine but also in the airway.

Mucin is produced in a wide range of host tissues including the gastrointestinal tract, lungs, kidneys, ovaries, breast, and pancreas. Under normal physiological conditions, mucin plays a protective role for epithelial tissues, functioning in the renewal and differentiation of the epithelium, and in the regulation of cell adhesion and effector cell function.

High-level expression of mucin is associated with metastasis and poor clinical outcome in patients diagnosed with cancer. Analysis of normal and tumor-derived mucins reveals that the carbohydrate chains of mucins are shorter and more sialylated in developing tumors compared to normal tissues. Also, unlike normal cells where mucin is expressed on the apical surface, in cancer cells, mucins are overexpressed over the entire cell surface. The synthesis of mucin on the surface of normal epithelial cells is under careful regulation, but in tumors there is an over-abundance of mucin due, in part, to elevated expression of MUC1. MUC1 is structurally unique, possessing a transmembrane domain and a larger extracellular domain made up of tandem repeats of 20 amino acids, and a cytoplasmic tail. The glycoprotein is present in as many as 30 to 100 cellular copies, and unlike high levels of soluble mucin (e.g., MUC2, MUC3, MUC5AC, MUC5B), MUC1 mucin is predominately associated with the cell membrane.

Since metastatic disease remains incurable, comprehensive efforts need to be undertaken to optimize present therapies. To accomplish this goal, factors contributing to sub-optimal tumor response must be identified and investigated. This application advances this goal by targeting mucin in cancer chemotherapy.

In addition, this application teaches methods of regulating mucin expression and/or secretion to improve accessibility of a substance to the cell surface and improve permeability of substances into the cell.

### SUMMARY

The invention provides methods and compositions for improving cellular uptake of an agent, such as a therapeutic agent, that is desired to be introduced into a cell. The present invention also provides methods and compositions for improving access of an agent to the cell surface. These methods and compositions are useful in the treatment of mucinous carcinomas characterized by an increased expression and/or secretion of mucins.

In one aspect, the invention provides a method for increasing or improving the uptake of an agent by a cell. The method comprises contacting the cell with a mucin inhibitor and/or a mucolytic agent. The cell can be contacted with the mucin inhibitor and/or the mucolytic agent prior to contacting the cell with the agent, or at substantially the same time as, contacting the cell with the agent. This method permits the agent to be taken up by the cell at a higher level than when the cell is not contacted with the mucin inhibitor and/or the mucolytic agent prior to contacting the cell with the agent.

In another aspect, a method for increasing the diffusion of an agent into a cell is provided. The method comprises contacting the cell with a mucin inhibitor and/or a mucolytic agent. The cell can be contacted with the mucin inhibitor and/or the mucolytic agent prior to contacting the cell with the agent, or at substantially the same time as, contacting the cell with the agent. This method increases the diffusion of an agent into a cell compared to diffusion of the agent into a cell that is not contacted with the mucin inhibitor and/or the mucolytic agent prior to contacting the cell with the agent.

In another aspect, the invention provides methods of increasing the permeability of an agent into a cell. The method comprises contacting the cell with a mucin inhibitor and/or a mucolytic agent. The cell can be contacted with the mucin inhibitor and/or the mucolytic agent prior to contacting the cell with the agent, or at substantially the same time as, contacting the cell with the agent. This method increases the permeability of the agent into a cell compared to the permeability of the agent into a cell that is not contacted with the mucin inhibitor and/or the mucolytic agent prior to contacting the cell with the agent. This method can be used to introduce or transfect an agent into a cell that expresses or secretes high levels of mucin. For example, nucleic acids, proteins, small molecules, and others can be introduced into a cell more effectively using this method compared to a method that does not involve contacting the cell with a mucin inhibitor and/or a mucolytic agent prior to contacting the cell with nucleic acids, proteins, or small molecules.

In yet another aspect, methods are provided that improve the ability of an agent to bind to, or access, the surface of a cell. For example, the methods improve the ability of an antibody to bind to a cell surface antigen, a ligand to bind to its cognate cell surface receptor, or a molecule to bind and enter a channel at the cell surface. The method comprises contacting the cell with a mucin inhibitor and/or a mucolytic agent. The cell can be contacted with the mucin inhibitor and/or the mucolytic agent prior to contacting the cell with the agent, or at substantially the same time as, contacting the cell with the agent. This method improves the ability of the agent to bind to, or access, the surface of a cell compared to the binding or access of the agent to the cell surface in the absence of treatment with a mucin inhibitor and/or a mucolytic agent.

In certain aspects, the invention provides methods of increasing the therapeutic activity of a therapeutic agent. The method involves contacting a cell with a mucin inhibitor and/or a mucolytic agent. This contacting step is performed prior to, or at substantially the same time as, contacting the cell with the therapeutic agent. This method increases or improves the therapeutic activity of the therapeutic agent compared to the therapeutic activity of the agent in a cell not contacted with the mucin inhibitor and/or the mucolytic agent. In a specific embodiment, these methods increase the cytotoxicity of a cytotoxic agent. In certain embodiments, the cytotoxic agent is selected from the group consisting of an alkylating agent, an anthracycline, a cytoskeletal disruptor, an epothilone, an inhibitor of topoisomerase II, a nucleoside analog and a precursor analog, a peptide antibiotic, a platinum-based agent, a retinoid, and a vinca alkaloid derivative. By improving the therapeutic activity of the agent, lower doses of the agent can be used to contact the cell, thereby decreasing any side effects from the use of the agent. In addition, the methods of the invention permit reducing the frequency of administration of a therapeutic agent.

In all of the above aspects, the methods can be performed in vitro or in vivo. In some embodiments of all the above aspects, the cell expresses or secretes higher levels of mucin than a normal cell of the same cell type. In other embodiments of all the above aspects, the cell expresses or secretes higher levels of mucin than other cell types. In certain other embodiments of all the above aspects, the cell expresses or secretes higher levels of mucin than a baseline level expression of mucin determined by comparing expression in normal cells versus cancerous cells. In some embodiments, of all of the above aspects, if a mucin inhibitor and a mucolytic agent are used to contact a cell, the mucin inhibitor and mucolytic agent can be administered before, after, or at substantially the same time as each other. In all of the above aspects, the method may involve contacting a cell with a mucin inhibitor and/or a mucolytic agent prior to, and subsequent to, contacting the cell with a therapeutic agent.

In another aspect, the invention provides methods of treating a subject having a mucinous carcinoma characterized by increased secretion and/or expression of extracellular mucin. In some embodiments, the expression and/or secretion of mucin is increased compared to other cells of the same cell type in a subject not having the mucinous carcinoma. In other embodiments of all the above aspects, the cell expresses or secretes higher levels of mucin than other cell types. In certain other embodiments of all the above aspects, the cell expresses or secretes higher levels of mucin than a baseline level expression of mucin determined by comparing expression in normal cells versus cancerous cells. The method involves administering to the subject a mucin inhibitor and/or a mucolytic agent prior to, or at substantially the same time as, administering a therapeutic agent useful in treating the underlying mucinous carcinoma to the subject. This method results in improved treatment of the mucinous carcinoma compared to treatment of the subject without the administration of a mucin inhibitor and/or a mucolytic agent. In specific embodiments, the mucinous carcinoma is selected from the group consisting of pancreatic cancer, prostate cancer, colon cancer, breast cancer, ovarian cancer, thyroid cancer, colorectal cancer, and lung cancer. In some embodiments, the mucin inhibitor and/or mucolytic agent is administered prior to, or at substantially the same time as, treatment with the therapeutic agent. In specific embodiments, the mucin inhibitor and/or mucolytic agent is administered between about 0.5 hr and about 96 hr before the administration of the therapeutic agent. In some embodiments, if a mucin inhibitor and a mucolytic agent are administered to a subject, the mucin inhibitor and mucolytic agent can be administered before, after, or at substantially the same time as each other. In other embodiments, the method may involve administering a subject with a mucin inhibitor and/or a mucolytic agent prior to, and subsequent to, administering the subject with a therapeutic agent. In some embodiments, the treatment further comprises administering to the subject at least one of: a multidrug transporter inhibitor, an N-glycosylation inhibitor, and a sialyltransferase inhibitor. In some embodiments, these substances may be administered at substantially the same time as the mucin inhibitor and/or mucolytic agent, or they may be administered prior to, or subsequent to, the mucin inhibitor and/or mucolytic agent. In certain embodiments, these substances are co-formulated with the mucin inhibitor and/or mucolytic agent. In other embodiments, these substances may be administered at substantially the same time as, or after the administration of the therapeutic agent.

In a further aspect, a method of improving chemotherapeutic treatment in a subject in need thereof is provided. The method comprises administering to the subject a mucin inhibitor and/or mucolytic agent prior to, or at substantially the same time as, the administration of a chemotherapeutic agent. As a result of this method, the subject has improved response to the treatment compared with a patient not administered a mucin inhibitor and/or mucolytic agent prior to the administration of a chemotherapeutic agent. In some embodiments, the chemotherapeutic agent is selected from the group consisting of an alkylating agent, an anthracycline, a cytoskeletal disruptor, an epothilone, an inhibitor of topoisomerase II, a nucleoside analog and a precursor analog, a peptide antibiotic, a platinum-based agent, a retinoid, a vinca alkaloid derivative, and combinations thereof. In specific embodiments, the mucin inhibitor and/or mucolytic agent is administered between about 0.5 hr and about 96 hr before the administration of the chemotherapeutic agent. In some embodiments, if a mucin inhibitor and a mucolytic agent are administered to a subject, the mucin inhibitor and mucolytic agent can be administered before, after, or at substantially the same time as each other. In other embodiments, the method may involve administering a subject with a mucin inhibitor and/or a mucolytic agent prior to, and subsequent to, administering the subject with a chemotherapeutic agent. In specific embodiments, the chemotherapeutic treatment is performed along with radiation therapy. In some embodiments, the method further comprises administering to the subject at least one of: a multidrug transporter inhibitor, an N-glycosylation inhibitor, and a sialyltransferase inhibitor. These substances may be administered at substantially the same time as the mucin inhibitor and/or mucolytic agent, or they may be administered prior to, or subsequent to, the mucin inhibitor and/or mucolytic agent. In certain embodiments, these substances are co-formulated with the mucin inhibitor and/or mucolytic agent. In other embodiments, these substances may be administered at substantially the same time as, or after the administration of the therapeutic agent.

In yet another aspect, the invention provides methods of improving drug therapy in a subject in need thereof. In certain embodiments, the subject has a mucinous carcinoma characterized by increased expression and/or secretion of mucin. The method involves administering to the subject a mucin inhibitor and/or a mucolytic agent. This administration is performed prior to, or at substantially the same time as, administering the drug that is useful in treating the underlying mucinous carcinoma of the subject. This method increases or improves the efficacy of the drug compared to the efficacy of the drug in a subject not administered with the mucin inhibitor and/or the mucolytic agent.

In another aspect, the invention provides compositions comprising a mucin inhibitor and/or a mucolytic agent and at least one of: a therapeutic agent, a multidrug transporter inhibitor, an N-glycosylation inhibitor, and a sialyltransferase inhibitor. In some embodiments, these substances are formulated together. In other embodiments they are provided as separate components as a kit. The kit may further comprise any materials that are useful in dispensing the substances provided in the kit, instructions for use, and any other materials that may be considered useful for inclusion.

In all of the above aspects, a mucin inhibitor includes any substance that decreases the expression and/or secretion of mucin. In specific embodiments, the mucin inhibitor is a mucin O-glycosylation inhibitor. In specific embodiments, the mucin inhibitor is selected from the group consisting of α-benzyl-GalNAc, Galβ1-4-Glc-Nacβ-O-naphthalenemethanol, Galβ1-3 GlcNAcβ-O-naphthalenemethanol, a UDP-Glc/GlcNAc C₄-epimerase inhibitor, and combinations thereof.

In all of the above aspects, a mucolytic agent includes any substance that non-specifically inhibits mucin expression or secretion. In some embodiments, the mucolytic agent is selected from the group consisting of guaifenesin, acetylcysteine, n-acety l-lcysteine, t-butylcysteine, fatty acid derivatives of cysteine, n-guanylcysteine, carbocysteine, ethyl cysteine, mecysteine, nesosteine, DNase, iodine, iodinated glycerol, potassium iodide, gelsolin, sodium 2-mercaptoethane sulphonate, bromheksin, erdosteine, tyloxapol, ipecacuanha, althea root, senega, antimony pentasulfide, creosote, guaiacolsulfonate, levoverbenone, bromhexine, eprazinone, mesna, ambroxol, sobrerol, domiodol, letosteine, stepronin, tiopronin, dornase alfa, neltenexine, erdosteine, mugwort, bromelain, papain, clerodendrum, gaseous nitrous oxide, and combinations thereof.

In all of the above aspects, the therapeutic agent is any substance that is considered by a clinician to be effective in treating a disease or condition.

### DESCRIPTION OF THE FIGURES

The foregoing and other objects of the present invention, the various features thereof, as well as the invention itself may be more fully understood from the following description, when read together with the accompanying drawings in which:

FIG. 1A is a representation of a Differential Interference Contrast (DIC) microscopy image of clusters of human pancreatic cancer cell line Capan-1.

FIG. 1B is a representation of a DIC microscopy image of clusters of human pancreatic cancer cell line HPAF-II.

FIG. 1C is a representation of a DIC microscopy image showing uniform arrangement of human brain cancer cell line U-87 MG.

FIG. 1D is a representation of a fluorescence microscopy image showing the relative extent of FITC-conjugated anti-MUC1 antibody (CD227) association with Capan-1 cells.

FIG. 1E is a representation of a fluorescence microscopy image showing the relative extent of FITC-conjugated anti-MUC1 antibody (CD227) association with HPAF-II cells.

FIG. 1F is a representation of a fluorescence microscopy image showing the relative extent of FITC-conjugated anti-MUC1 antibody (CD227) association with U-87 MG cells.

FIG. 1G is a representation of a superimposed image of the DIC and fluorescent images of FIGS. 1A and 1D showing the localization of antibody with respect to Capan-1 cellular clusters (20× magnification).

FIG. 1H is a representation of a superimposed image of the DIC and fluorescent images of FIGS. 1B and 1E showing the localization of antibody with respect to HPAF-II cellular clusters (20× magnification).

FIG. 1I is a representation of a superimposed image of the DIC and fluorescent images of FIGS. 1C and 1F showing the localization of antibody with respect to U-87 MG cellular clusters (20× magnification).

FIG. 2A is a graphic representation of percent cell viability of Capan-1 (◯), HPAF-II (▪) and U-87 MG (Δ) cells determined after exposure of 1×10⁴ cells/ml to different concentrations of benzyl-α-GalNAc for 72 hr.

FIG. 2B is a graphic representation of real time RT-PCR MUC1 mRNA expression levels for Capan-1, HPAF-II and U-87MG cells.

FIG. 2Ca is a representation of a DIC microscopy image of Capan-1 cells not exposed to benzyl-α-GalNAc.

FIG. 2Cb is a representation of a DIC microscopy image of HPAF-H cells not exposed to benzyl-α-GalNAc.

FIG. 2Cc is a representation of a DIC microscopy image of U87-MG cells not exposed to benzyl-α-GalNAc.

FIG. 2Cd is a representation of a DIC microscopy image of Capan-1 cells exposed to 0.4 mg/ml of benzyl-α-GalNAc for 72 hr.

FIG. 2Ce is a representation of a DIC microscopy image of HPAF-II cells exposed to 0.8 mg/ml of benzyl-α-GalNAc for 72 hr.

FIG. 2CF is a representation of a DIC microscopy image of U87-MG cells exposed to 0.8 mg/ml of benzyl-α-GalNAc for 72 hr.

FIG. 3A is a graphic representation of the fluorescence intensities for antibody associated with MUC1 mucin after Capan-1 cells were exposed to benzyl-α-GalNAc for 24 hr, 48 hr, and 72 hr followed by 24 hr of incubation with FITC-conjugated anti-MUC1 (CD227) monoclonal antibody (4 μl/well) at 37° C. The relative fluorescence intensities correlate with amount of antibody associated with cells exposed to benzyl-α-GalNAc (+) and without exposure to benzyl-α-GalNAc (−). Fluorescence was measured at excitation wavelength of 485 nm and emission wavelength of 528 nm.

FIG. 3B is a graphic representation of the fluorescence intensities for antibody associated with MUC1 mucin after HPAF-II cells were exposed to benzyl-α-GalNAc for 24 hr, 48 hr, and 72 hr followed by 24 hr of incubation with FITC-conjugated anti-MUC1 (CD227) monoclonal antibody (4 μl/well) at 37° C.

FIG. 3C is a graphic representation of a fluorescence-activated cell sorting analysis for Capan-1 cells exposed to benzyl-α-GalNAc as compared to cells not exposed to benzyl-α-GalNAc.

FIG. 3D is a graphic representation of a fluorescence-activated cell sorting analysis for HPAF-II cells exposed to benzyl-α-GalNAc as compared to cells not exposed to benzyl-α-GalNAc.

FIG. 3E is a graphic representation of a fluorescence-activated cell sorting analysis for U-87 MG cells exposed to benzyl-α-GalNAc as compared to cells not exposed to benzyl-α-GalNAc.

FIG. 4A is a graphic representation of the percent viability of HPAF-II cells after 1×10⁴ cells/ml were exposed to benzyl-α-GalNAc for 48 hr followed by a 24 hr treatment with 5-FU.

FIG. 4B is a graphic representation of the percent viability of Capan-1, HPAF-II, and U-87 MG cells exposed to benzyl-α-GalNAc followed by 5-FU treatment as compared to cells treated with 5-FU alone.

FIG. 5A is a graphic representation of percent viability of Capan-1 cells exposed to 0.4 mg/ml of benzyl-α-GalNAc for 48 hr followed by a wash with 1×PBS and grown for the next 24 hr, 48 hr, and 72 hr in fresh media. The percent viability of cells was measured at each time point following exposure to benzyl-α-GalNAc (open bars, □) and compared with percent viability of cells not exposed to benzyl-α-GalNAc (closed bars, ▪).

FIG. 5B is a graphic representation of percent viability of HPAF-II cells exposed to 0.8 mg/ml of benzyl-α-GalNAc for 48 hr followed by a wash with 1×PBS and grown for the next 24 hr, 48 hr, and 72 hr in fresh media. The percent viability of cells was measured at each time point following exposure to benzyl-α-GalNAc (open bars, □) and compared with percent viability of cells not exposed to benzyl-α-GalNAc (closed bars, ▪).

FIG. 5C is a representation of a DIC microcopy image of Capan-1 cells described in FIG. 5A (20× magnification).

FIG. 5D is a representation of a DIC microcopy image of HPAF-II cells described in FIG. 5B (20× magnification).

FIG. 6A is a graphic representation of percent viability of HPAF-II cells grown at increasing concentrations of neuraminidase for 1 hr at 37° C.

FIG. 6B is a representation of a FACS analysis showing a decrease in fluorescence intensity (correlating to association of FITC-conjugated MAA lectin to the sialic acid residues) for cells exposed to (+) neuraminidase compared to cells without exposure (−) to neuraminidase.

FIG. 6C is a representation of a FACS analysis showing no change in fluorescence peaks for CD227 association with or without neuraminidase treatment.

FIG. 6D is a graphic representation of the percent cell viability observed after 5-FU treatment between cells following exposure to neuraminidase (+) or without exposure to neuraminidase (−).

FIG. 7Aa is a representation of a fluorescent image of the immuno-histochemical staining of Capan-1 tumor specimens using an anti-MUC1 antibody (NCL-MUC1). The figure is shown at a 20× magnification and the bar corresponds to 50 μm.

FIG. 7Ab is a representation of a fluorescent image of the immuno-histochemical staining of HPAF-II tumor specimens using an anti-MUC1 antibody (NCL-MUC1). The figure is shown at a 20× magnification and the bar corresponds to 50 μm.

FIG. 7Ac is a representation of a fluorescent image of the immuno-histochemical staining of U-87 MG tumor specimens using an anti-MUC1 antibody (NCL-MUC1). The figure is shown at a 20× magnification and the bar corresponds to 50 μm.

FIG. 7Ba is a representation of a fluorescent image of pancreatic HPAF-II tumor cells that were treated with saline injections (control) instead of benzyl-α-GalNAc. The figure is shown at a 20× magnification and the bar corresponds to 50 μm.

FIG. 7Bb is a representation of a fluorescent image of pancreatic HPAF-H tumor cells that were treated with benzyl-α-GalNAc. These tumors received four intratumoral injections of benzyl-α-GalNAc (10 mg/ml, 0.1 cc) at intervals of 48 hr. The figure is shown at a 20× magnification and the bar corresponds to 50 μm.

FIG. 7Bc is a representation of a fluorescent image of pancreatic Capan-1 tumor cells that were treated with saline injections (control) instead of benzyl-α-GalNAc. The figure is shown at a 20× magnification and the bar corresponds to 50 μm.

FIG. 7Bd is a representation of a fluorescent image of pancreatic Capan-1 tumor cells that were treated with benzyl-α-GalNAc. These tumors received four intratumoral injections of benzyl-α-GalNAc (10 mg/ml, 0.1 cc) at intervals of 48 hr. The figure is shown at a 20× magnification and the bar corresponds to 50 μm.

FIG. 8A is a graphic representation of the body weight of animals monitored during the course of therapy. Capan-1 tumors were established in subcutaneous dorsa of female SCID mice. When the tumor size reached approximately 50-70 mm³, intratumoral injections of benzyl-α-GalNAc (0.1 ml, 10 mg/ml) were administered on days 4, 6, 8, and 10 while the control groups received comparable injections of saline. 5-FU therapy (arrow) began when the tumor size was approximately 100 mm³.

FIG. 8B is a graphic representation of the Capan-1 tumor volumes in animals exposed to saline, 5-FU, Benzyl-α-GalNAc, and Benzyl-α-GalNAc+5-FU over time.

FIG. 8Ca is a representation of a fluorescent image of Capan-1 tumor sections showing the arrangement of neoplastic cells within tumors treated with saline. The figure is shown at a 20× magnification and the bar corresponds to 50 μm.

FIG. 8Cb is a representation of a fluorescent image of Capan-1 tumor sections showing the arrangement of neoplastic cells within tumors treated with benzyl-α-GalNAc. The figure is shown at a 20× magnification and the bar corresponds to 50 μm.

FIG. 8Cc is a representation of a fluorescent image of Capan-1 tumor sections showing the arrangement of neoplastic cells within tumors treated with 5-FU. The figure is shown at a 20× magnification and the bar corresponds to 50 μm.

FIG. 8Cd is a representation of a fluorescent image of Capan-1 tumor sections showing the arrangement of neoplastic cells within tumors treated with benzyl-α-GalNAc+5-FU. The figure is shown at a 20× magnification and the bar corresponds to 50 μm.

FIG. 9A is a representation of DIC microscopy images (left panel), fluorescent microscopy images (middle panels), and merged images of the DIC and fluorescent microscopy images (right panels) depicting the intracellular uptake of 5-FU by Capan-1 cells after 1 hr of exposure to 5-FU. The Capan-1 cells were either treated with benzyl-α-GalNAc (+) or not (−) 48 hr prior to treatment with 5-FU.

FIG. 9B is a representation of DIC microscopy images (left panels), fluorescent microscopy images (middle panels), and merged images of the DIC and fluorescent microscopy images (right panels) depicting the intracellular uptake of 5-FU by Capan-1 cells after 4 hr of exposure to 5-FU. The Capan-1 cells were either treated with benzyl-α-GalNAc (+) or not (−) 48 hr prior to treatment with 5-FU.

FIG. 10A is a representation of DIC microscopy images (left panels), fluorescent microscopy images (middle panels), and merged images of the DIC and fluorescent microscopy images (right panels) depicting the intracellular uptake of 5-FU by U-87 MG cells after 1 hr of exposure to 5-FU. The U-87 MG cells were either treated with benzyl-α-GalNAc (+) or not (−) 48 hr prior to treatment with 5-FU.

FIG. 10B is a representation of DIC microscopy images (left panels), fluorescent microscopy images (middle panels), and merged images of the DIC and fluorescent microscopy images (right panels) depicting the intracellular uptake of 5-FU by U-87 MG cells after 4 hr of exposure to 5-FU. The U-87 MG cells were either treated with benzyl-α-GalNAc (+) or not (−) 48 hr prior to treatment with 5-FU.

FIG. 11 is a schematic representation of a model illustrating the enhanced intracellular accumulation of 5-FU following reduction of the mucin glycation mesh.

### DETAILED DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in case there is any inconsistency between the patents, patent applications, and publications, and this disclosure.

### DEFINITIONS

Unless defined otherwise, technical and scientific terms used herein have the same meaning as commonly understood by one of ordinary skill in the art to which this invention belongs. For purposes of the present invention, the following terms are defined below.

The term “mucin inhibitor” means a substance that inhibits any aspect of mucin expression or secretion through a specific mechanism of action. For example, a mucin inhibitor can prevent the formation of carbohydrate chains attached to the mucin protein core which eventually forms a mucin (glycation) mesh. Alternatively, a mucin inhibitor can inhibit the expression of a mucin gene, or the post-translational modification or secretion of a mucin protein.

The term “mucolytic agent” means a substance that, through a non-specific mechanism of action, prevents the formation of carbohydrate chains attached to the mucin protein core (e.g., MUCINEX®).

The term “agent” means any substance that is desired to be provided to a cell. The substance may be directed within the cell or to the cell surface.

The term “substance” means that which has mass and occupies space.

The term “subject” encompasses any animal, such as a mammal. The term “mammal” means any animal classified as a mammal, including humans, domestic animals (e.g., dogs, cats), zoo animals, farm animals (e.g., cattle, horses, sheep, pigs, goats, rabbits), as well as rodents, such as mice and rats, etc.

The term “therapeutic agent” means any substance that is effective in treating a mucinous carcinoma or reducing the risk of developing a mucinous carcinoma.

The term “treating” means the reduction or amelioration of any medical disorder to any extent, and includes, but does not require, a complete cure of the disorder.

The term “inhibit” refers to the act of diminishing, suppressing, alleviating, preventing, reducing or eliminating. The term “reduce” means to decrease to any extent.

The term “contacting” encompasses any mode of interaction between a substance and an object being contacted (e.g., a cancer cell). The interaction of the substance with the object being contacted can occur at substantially the same time as the time of administration of the substance, over an extended period of time starting from around the time of administration of the substance, or be delayed from the time of administration of the substance.

The term “mucinous carcinoma” refers to tumors that secrete moderate to high levels of mucin. The determination of the level of mucin secretion as moderate or high is based on a comparison with the level of secretion in a normal cell from which the tumor arose, or a baseline level of expression determined for the cell type from which the tumor arose.

The term “an effective amount” means an amount of a substance that elicits a response in a subject that is being sought by the doctor, other clinician, veterinarian, or researcher.

The term “about” means 20% around a numerical value. For example, “about 10 hr” means 8 hr to 12 hr; and “about 100 mg” means 80 mg to 120 mg.

The term “at substantially the same time,” when used in the context of administration of two agents, means that one agent is administered at the same time as the other or no later than about 10 min before the other agent.

### Role of Reduction of Mucin Glycosylation in Disease

The present invention arose, in part, from an attempt to understand why therapeutic agents used in certain mucinous carcinomashave had limited clinical benefits. For example, in the case of pancreatic cancer, the popular chemotherapeutic approaches of using gemcitabine and/or fluorouracil have only been modestly effective in reducing tumor growth. The inventors have found that this reduced effectiveness is related, in part, to the expression and/or secretion of the highly glycosylated protein mucin. More specifically, the inventors have found that reducing the levels of mucin glycosylation by using O-glycosylation inhibitors in mucinous carcinomas characterized by increased expression and/or secretion of mucin can improve the therapeutic effectiveness of drugs used to treat the underlying mucinous carcinoma. Accordingly, the present invention relates, in part, to methods of improving the effectiveness of therapeutic agents; methods of increasing cellular uptake of a therapeutic agent; methods of improving access to the cell surface; methods of improving the effectiveness of chemotherapy; methods of reducing the dose of a therapeutic agent for treating a mucinous carcinoma; and methods of treating a mucinous carcinoma characterized by deregulated expression and/or secretion of mucins. The invention also relates to compositions and kits comprising a mucin inhibitor and/or mucolytic agent and a therapeutic agent.

A. Mucin Inhibitors and Mucolytic Agents

Mucin is a component of mucus, the clear viscous secretion of the mucus membranes. It is a carbohydrate-rich glycoprotein that is secreted by specialized epithelial cells known as goblet cells, the submaxillary glands, and other mucus glandular cells. Goblet cells are specialized for secretion and contain an accumulation of mucus secretory granules. Mucus tissue (or mucosa) lines various anatomic structures in the mammalian and avian body, including the eyes, respiratory tract (alveoli, bronchi, oral cavity, larynx, nasal cavity, pharynx, trachea), gastrointestinal tract (esophagus, stomach, small and large intestine, rectum), and genitourinary tract (urethra, urinary bladder, uterus, and vagina). Alterations in the quantity of mucus secretions may be due to various underlying factors, including a change in the amount of mucus glycoproteins secreted from mucus-secreting cells, a change in the total number of mucus-secreting cells, or combinations thereof. Mediators released by the inflammatory response are known to act as mucus secretagogues, including lipid mediators, oxygen metabolites, and other cell-specific products (Larivee et al., in Airway Secretion, Takishima and Shimura (Eds.), Marcel Dekker Inc., 1994, pages 469-511).

Methods of regulating mucin expression and/or secretion depend upon the development of mucin inhibitors. Inhibitors of the enzymes involved in glycosylation are one group of mucin inhibitors that are likely to be important for new and effective therapeutic strategies. Mucin inhibitors target different aspects of mucin synthesis. Some mucin inhibitors reduce O-glycosylation levels which reduces the glycation mesh. In these cases, the mucin core remains intact and is not influenced by the mucin inhibitor, but the mucin protein core by itself is not a barrier to drugs that are to enter the cell and/or antibodies/ligands that are to bind or access the cell surface.

Competitive substrate-based primers such as, but not limited to, α-benzyl-GalNAc, Galβ1-4-Glc-Nacβ-O-naphthalenemethanol, and Galβ1-3 GlcNAcβ-O-naphthalenemethanol, inhibit the extension of O-linked glycans on cells resulting in truncated O-linked glycans on cells. These inhibitors do not affect the attachment of GalNAc to threonine or serine residues of the substrate.

A different class of mucin inhibitors inhibit O-linked glycosylation by interfering with the biosynthesis of UDP-GalNAc, the nucleotide donor utilized by the enzymes N-acetyl-α-galactosaminyltransferases (ppGalNAcTs). Non-limiting examples of such inhibitors include the selective UDP-Glc/GlcNAc C₄-epimerase inhibitor which has a Kᵢ value of 11 μm (Winans et al., Chem Biol., 9:113-129 [2002]); and the inhibitors from a uridine library with Kᵢ value of ˜8.0 μm described in Hang et al., Chem Biol., 11:337-345 [2004]; and Hang et al., Bioorg. & Med. Chem., 13:5021-5034 [2005].

Yet another group of mucin inhibitors are based on benzyl-O—N-acetyl-D-galactosamine. Benzyl-O—N-acetyl-D-galactosamine is an inhibitor of the biosynthesis of mucin type O-gycans acting as a competitive inhibitor of the monosaccharide-protein glycosidic linkage, GalNAc-α-O-Ser/Thr. Non-limiting examples of such inhibitors are described in Patsos et al., Mol. Biol. Col. Cancer, 33(4):721-723 [2005]. Any O-glycosylation inhibitor that reduces or eliminates mucin levels through specific and/or non-specific mechanisms is envisioned as being part of the invention.

In addition to mucin O-glycosylation inhibitors, other inhibitors that decrease mucin synthesis or levels, or decrease in some way the over-production of mucin are also part of the present invention. These inhibitors include, for example, inhibitors of mucin gene expression, mucin protein post-translational modification, and/or mucin secretion. Such inhibitors include, but are not limited to, small molecule inhibitors, antisense, siRNA, and/or ribozyme inhibitors. Non-limiting examples include inhibitors of the ICACC chloride channel and the related channels described in WO99/44620, analogues and derivatives of anthranilic acid, analogues and derivatives of 2-amino-nicotinic acid, analogues and derivatives of 2-amino-phenylacetic acid, bendroflumethiazide, and prodrugs of any of these inhibitors. Some other non-limiting examples of mucin inhibitors that may be used in this invention include those disclosed in U.S. Pat. No. 6,737,427 and WO 2004/043392. Other examples of mucin inhibitors include antisense, siRNA, aptamers, and/or ribozyme inhibitors that directly target the expression of mucin genes that encode mucins expressed on the cell surface either by inhibiting regulators of the mucin genes or the transcript of the mucin gene itself. In some embodiments, the mucin inhibitor is an antibody or an antigen-binding fragment thereof. In addition, these inhibitors may also inhibit enzymes that post-translationally modify mucins.

Non-limiting examples of mucolytic agents for use in the present invention include guaifenesin (marketed as MUCINEX®), acetylcysteine, n-acetyl l-cysteine, t-butylcysteine, fatty acid derivatives of cysteine, n-guanylcysteine, carbocysteine, ethyl cysteine, mecysteine, nesosteine, DNase, iodine, iodinated glycerol, potassium iodide, gelsolin, sodium 2-mercaptoethane sulphonate, bromheksin, erdosteine, tyloxapol, ipecacuanha, althea root, senega, antimony pentasulfide, creosote, guaiacolsulfonate, levoverbenone, bromhexine, eprazinone, mesna, ambroxol, sobrerol, domiodol, letosteine, stepronin, tiopronin, dornase alfa, neltenexine, erdosteine, mugwort, bromelain, papain, clerodendrum, and gaseous nitrous oxide.

B. Therapeutic Agents

The therapeutic agents of the invention encompass any substance that is effective in treating a mucinous carcinoma or reducing the risk of a subject developing a mucinous carcinoma.

For example, therapeutic agents that are useful in the treatment of mucinous carcinomas include, without limitation, anti-inflammatory agents, or antiphlogistics. Antiphlogistics include, for example, glucocorticoids, such as cortisone, hydrocortisone, prednisone, prednisolone, fluorcortolone, triamcinolne, methylprednisolone, prednylidene, paramethasone, dexamethasone, betamethasone, beclomethasone, fluprednylidene, desoxymethasone, fluocinolone, flunethasone, diflucortolone, clocortolone, clobetasol and fluocortin butyl ester; immunosuppressive agents such as anti-TNF agents (e.g., etanercept, infliximab) and IL-1 inhibitors; penicillamine; non-steroidal anti-inflammatory drugs (NSAIDs) which encompass anti-inflammatory, analgesic, and antipyretic drugs such as salicyclic acid, celecoxib, difunisal and from substituted phenylacetic acid salts or 2phenylpropionic acid salts, such as alclofenac, ibutenac, ibuprofen, clindanac, fenclorac, ketoprofen, fenoprofen, indoprofen, fenclofenac, diclofenac, flurbiprofen, piprofen, naproxen, benoxaprofen, carprofen and cicloprofen; oxican derivatives, such as piroxican; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; nabunetone; and disease-modifying antirheumatic drugs (DMARDs) such as methotrexate, gold salts, hydroxychloroquine, sulfasalazine, cyclosporin, azathioprine, and leflunomide. Other exemplary therapeutic agents useful in the treatment of mucinous carcinomas include antioxidants such as, superoxide dismutase (SOD), 21-aminosteroids/aminochromans, vitamin C or E.

Other therapeutic agents useful in treating mucinous carcinomas include, but are not limited to, anti-angiogenic factors, chemotherapeutics, and radiomimetics.

Non-limiting examples of angiogenic factors include fibroblast growth factor (FGF), vascular endothelial growth factor (VEGF), placental growth factor (PlGF), Heparin-binding EGF-like growth factor, hepatocyte growth factor (HGF), transforming growth factor-beta (TGF-beta), interferon-γ (IFN-γ), interferon-γ-inducible protein-10 (IP-10), platelet-derived growth factor (PDGF), pleiotrophin, platelet factor-4 (PF-4), macrophage inflammatory protein-1 (MIP-1), and macrophage inflammatory protein-2 (MIP-2). Any inhibitor of an angiogenic factor may be used within the scope of this invention. Examples of inhibitors of angiogenesis include, without limitation, neutralizing antibodies, soluble receptors, aptamers, ribozymes, and siRNA molecules that target and interfere with the biological activity of an angiogenic factor.

In one embodiment, the angiogenic factor is vascular endothelial growth factor (VEGF). VEGF is a secreted cytokine that is structurally related to platelet derived growth factor (PDGF) and promotes tumor angiogenesis. The gene for VEGF undergoes alternative splicing to produce several isoforms including VEGF₁₂₁, VEGF₁₄₅, VEGF₁₆₅, VEGF₁₈₃, VEGF₁₈₉, and VEGF₂₀₆. A VEGF inhibitor may inhibit one or more of these isoforms. In some embodiments, the VEGF inhibitor targets VEGF₁₆₅. VEGF inhibitors include, but are not limited to, a neutralizing antibody against VEGF or its receptor, a soluble VEGF receptor that acts as a decoy receptor for VEGF, a small molecule tyrosine kinase inhibitor of VEGF receptors, or a ribozyme that specifically targets VEGF mRNA. Examples of VEGF inhibitors include, without limitation, pegaptanib sodium, ranibizumab, bevacizumab, HuMV833, squalamine lactate, anecortave acetate, triamcinolone acetonide, VEGF-Trap, tryptophanyl-tRNA synthetase, a stabilized ribozyme that targets the pre-mRNA of VEGFR-1 (Angiozyme™), and oral VEGF inhibitors such as, but not limited to, SU6668, SU11248, ZD6474, PTK787/ZK222584 (Vatalanib), and BAY 43-9006 (see, Cardones et al., Current Pharm. Design, 12:387-94 [2006]).

In another embodiment, the angiogenic factor is placental growth factor (PlGF). Non-limiting examples of anti-PlGF agents include anti-PlGF antibodies and aptamers that bind and prevent PlGF action.

Non-limiting examples of chemotherapeutic agents include alkylating agents, anthracyclines, cytoskeletal disruptors, epothilones, inhibitor of topoisomerase II, nucleoside analogs and precursor analogs, peptide antibiotics, platinum-based agents, retinoids, and vinca alkaloids and derivatives.

Non-limiting examples of alkylating agents include cyclophosphamide, mechlorethamine, chlorambucil, and melphalan. Anthracyclines include, but are not limited to, daunorubicin, doxorubicin, epirubicin, idarubicin, mitoxantrone, and valrubicin.

Non-limiting examples of cytoskeletal disruptors include paclitaxel and docetaxel. Examples of epothilones, include, but are not limited to, epothilone A, epothilone B, and epothilone D.

Non-limiting examples of topisomerase II inhibitors include etoposide, teniposide, and tafluposide. Nucleoside analogs and precursor analogs include, but are not limited to, azacitidine, azathioprine, capecitabine, cytarabine, doxifluridine, fluorouracil, gemcitabine, mercaptopurine, methotrexate, and tioguanine.

A non-limiting example of a peptide antibiotic is bleomycin. Platinum-based agents include, but are not limited to, carboplatin, cisplatin, and oxaliplatin.

A non-limiting example of a retinoid is all-trans retinoic acid. Vinca alkaloids include, but are not limited to, vinblastine, vincristine, vindesine, and vinorelbine.

Examples of radiomimetics for use in the present invention, include, but are not limited to, mustine, uramustine, cyclophosphamide, chlorambucil, and melphalan.

In a specific embodiment, in the treatment of a pancreatic cancer, a prostate cancer, a colon cancer, a breast cancer, an ovarian cancer, a lung cancer, and a thyroid cancer, the therapeutic agent is selected from the group consisting of etoposide, paclitaxel, camptothecin, lomustine, teniposide, and any fluoropyrimidine (e.g., 5-FU, FUrd, FUMP, FdUMP). In some cases, in the treatment of pancreatic cancer, for example, one or more therapeutic agents is/are provided to a cell using PEGylated cationic liposomes (PCLs) (Kalra and Campbell, Pharm. Res., 23(12):2809-2817 [2006]).

C. Methods of Improving Access, Cellular Uptake, and Effectiveness of a Therapeutic Agent

It has been determined that increased expression of mucins reduce the therapeutic effectiveness of drugs in subjects having mucinous carcinomas characterized by increased expression and/or secretion of mucin. This decreased therapeutic effectiveness of a drug may, in part, be due to decreased access of therapeutic agents to the cell surface.

In normal cells, mucins are only expressed on the apical surface; however, in many cancer cells, mucin expression is found over the entire surface of the cell. For example, in pancreatic cancer cell lines, the mucin MUC1, is highly expressed on the cell surface. Exposure of pancreatic cells to a mucin inhibitor was found to increase the ability of antibodies raised to the mucin core to bind to the pancreatic cells compared with cells not treated with a mucin inhibitor. This is likely due to the mucin inhibitor's action in reducing the mucin mesh on the cell surface and improving the general access of the antibody to the MUC1 antigen. Thus, the present invention provides a method for increasing the access of a substance to the surface of a cell that expresses high levels of mucin. The level of mucin is determined to be “high” compared to the normal level of expression of mucin on the surface of the cell type in question, or compared to other cells that are able to permit access of the substance to the cell surface. By contacting a cell expressing or secreting high levels of mucin with a mucin inhibitor and/or a mucolytic agent prior to contacting the cell with a desired substance, the desired substance has greater access to the cell surface than without the cell being contacted with the mucin inhibitor and/or the mucolytic agent. This method permits an improvement in the ability of antibodies to bind their cell surface targets, for ligands to bind their receptors, and for other molecules to be able to pass through channels in the cell membrane compared with a method that does not involve contacting of the cell with a mucin inhibitor and/or a mucolytic agent.

According to aspects of the present invention, in those cells having increased mucin expression and/or secretion, the permeability and/or diffusion of a therapeutic agent across the mucus layer can also be enhanced by contacting the cell with a mucin inhibitor and/or a mucolytic agent prior to administration of the therapeutic agent. Examples of cells with increased mucin expression and/or secretion include, but are not limited to, pancreatic cancer cells, colon cancer cells, thyroid cancer cells, prostate cancer cells, lung cancer cells, breast cancer cells, and ovarian cancer cells. The cells can be contacted with a mucin inhibitor and/or a mucolytic agent at any time prior to contacting the cells with a therapeutic agent. In some embodiments, the cells are contacted with a mucin inhibitor and/or a mucolytic agent about 0.5 hr, about 1 hr, about 2 hr, about 3 hr, about 4 hr, about 5 hr, about 6 hr, about 12 hr, about 24 hr, about 48 hr, or about 96 hr before contact with the therapeutic agent. In other embodiments, the cells are contacted with a mucin inhibitor or a mucolytic agent about 0.5 hr to about 6 hr, about 0.5 hr to about 12 hr, about 0.5 hr to about 24 hr, about 0.5 hr to about 48 hr, about 0.5 hr to about 96 hr, about 2 hr to about 8 hrs, about 4 hr to about 12 hr, about 12 hr to about 24 hr, about 24 hr to about 48 hr, or about 48 hr to about 96 hr before contact with the therapeutic agent. In certain embodiments, the cells are contacted with a mucin inhibitor and/or a mucolytic agent at substantially the same time as the cells are contacted with the therapeutic agent. In certain cases where both a mucin inhibitor and a mucolytic agent are used to contact a cell, the mucin inhibitor and mucolytic agent are used to contact a cell at the same time or at different times. In specific embodiments, the mucin inhibitor is used to contact the cell prior to the mucolytic agent. In another embodiment, the mucolytic agent is used to contact the cell prior to the mucin inhibitor. In yet other embodiments, mucin inhibitor and/or mucolytic agent may be used both to contact a cell prior to, and after, contacting the cell with a therapeutic agent.

To be effective, most therapeutic agents should be able to enter a cell and be present in a sufficient amount within the cell. Contacting cells expressing or secreting high levels of mucin with a mucin inhibitor and/or a mucolytic agent prior to, or at substantially the same time as, administration of a therapeutic agent can increase the intracellular uptake of the therapeutic agent. For example, when pancreatic cancer cells were exposed to the chemotherapeutic agent, 5-fluorouracil (5-FU), about an hour after contacting the cells with a mucin inhibitor, 5-FU was found at high intracellular levels within the cancer cells. By increasing the uptake of a therapeutic agent, the therapeutic agent can manifest its function(s) more effectively. In fact, in a pancreatic tumor model, inhibiting mucin O-glycosylation prior to treatment with 5-FU was found to increase the anti-tumor activity of 5-FU.

By increasing the cellular uptake of a therapeutic agent, the use of a mucin inhibitor and/or a mucolytic agent prior to, or at substantially the same time as, administration of the therapeutic agent, permits administration of lower doses of a therapeutic agent, thereby decreasing the toxicity and other side effects of the therapeutic agent, while maintaining the effectiveness of the therapeutic agent. In addition, because of its increased uptake and therefore effectiveness, the methods of the invention permit a reduction in the frequency of administration of a therapeutic agent.

Cells which have acquired resistance to chemotherapeutic drugs may show altered levels of mucins. For example, long term exposure of HT-29 colon cancer cells to 5-FU and methotrexate resulted in the differentiation of these cells to a relatively high mucin-secreting phenotype without an alteration in the levels of the multidrug resistant p-glycoprotein. The use of a mucin inhibitor and/or a mucolytic agent prior to contact with the chemotherapeutic agent can improve the effectiveness of a chemotherapeutic agent in “resistant” cells (i.e., cells that are no longer effectively treated with a chemotherapeutic agent). The inhibition of mucin may reduce the mucin mesh and facilitate the diffusion of the chemotherapeutic agent across the compromised mucus layer, improving the intracellular drug uptake and enhancing the cytotoxic effects of the drug.

### Methods of Treatment

Increased mucin production occurs in many adenocarcinomas, including cancer of the pancreas, lung, thyroid, breast, ovary, and colon. Mucins are expressed at different levels in these cancers. The relative abundance of different types of mucins overexpressed in tumors depends upon the origin of the tumor. For example, colon carcinomas overexpress MUC5AC, MUC6, and MUC17; lung adenocarcinomas express high levels of MUC1, MUC3, and MUC4; and MUC1 and MUC4 are expressed at high levels in prostate and pancreatic cancers.

Aspects of the present invention provides methods of treating a subject diagnosed with any mucinous carcinoma characterized by increased mucin expression and/or secretion. The method comprises administering to the subject an effective amount of a mucin inhibitor and/or a mucolytic agent prior to, or at substantially the same time as, administration of a therapeutic agent that is useful in treating the underlying mucinous carcinoma. The administration of the mucin inhibitor and/or the mucolytic agent increases the intracellular uptake of the therapeutic agent and increases the effectiveness of the agent. By the methods of the invention, therapeutic agents can be administered at lower doses to the subject thereby decreasing any side effects associated with increased doses of the therapeutic agent while maintaining its efficacy. Non-limiting examples of mucinous carcinomas that can be treated according to the present invention include, pancreatic cancer, prostate cancer, colon cancer, breast cancer, ovarian cancer, thyroid cancer, and lung cancer. In one example, a subject in need of treatment for a mucinous carcinoma characterized by increased mucin expression and/or secretion is administered a mucin inhibitor prior to treatment with the therapeutic agent. In another example, a subject in need of treatment for a mucinous carcinoma characterized by increased mucin expression and/or secretion is administered a mucolytic agent prior to treatment with the therapeutic agent. In other examples, the subject in need of treatment for a mucinous carcinoma characterized by increased mucin expression and/or secretion is administered a combination of a mucin inhibitor and a mucolytic agent prior to treatment with the therapeutic agent. In certain cases where both a mucin inhibitor and a mucolytic agent are administered to a subject, the mucin inhibitor and mucolytic agent are administered at the same time, or at different times. In specific embodiments, the mucin inhibitor is administered to the subject prior to the mucolytic agent. In another embodiment, the mucolytic agent is administered to the subject prior to the mucin inhibitor. In some cases, a mucin inhibitor and/or a mucolytic agent is administered both prior to, and after, administration of the therapeutic agent to the subject.

In the context of chemotherapy, aspects of the present invention provide methods for increased efficacy of the chemotherapeutic or radiomimetic agent. A cancer patient may be treated with a mucin inhibitor and/or a mucolytic agent prior to, or at substantially the same time as, administration of a chemotherapeutic or radiomimetic agent. This process increases the uptake of the chemotherapeutic agent by a cancerous cell and with the presence of higher levels of the agent within the cancerous cell (compared to without prior treatment with a mucin inhibitor and/or a mucolytic agent), and leads to a better therapeutic effectiveness (i.e., by increased killing of cancerous cells). In some examples, the mucin inhibitor and/or mucolytic agent are/is directly administered into the tumor. In certain examples, the mucin inhibitor and/or mucolytic agent are/is provided via a sustained release device so that the mucin inhibitor and/or mucolytic agent are/is present before and during the entire span of the chemotherapeutic or radiomimetic treatment. In certain instances, the mucin inhibitor/therapeutic agent treatment can be combined with and/or followed by radiation therapy.

The methods of treatment also encompass the use of N-glycosylation inhibitors and/or sialyltransferase inhibitors in combination with a mucin inhibitor or a mucolytic agent to treat a mucinous carcinoma characterized by increased expression or secretion of mucin. Non-limiting examples of N-glycan inhibitors include deoxymannojiramicin, swainsonine, tunicamycin, and castanospermine. Non-limiting examples of sialyltransferase inhibitors include neuraminidase, lithocholic acid analogs, and others for example described in Wang et al., Med. Res. Rev., 23(1):32-47 (2003) and Drinnan et al., Mini Rev. Med. Chem., 3(6):501-517 (2003).

Many mammalian cells express membrane proteins that transport large hydrophobic molecules (e.g., anti-cancer drugs, antibiotics, steroids, calcium channel blockers, and opioids) out of the cells. These membrane proteins are called “multidrug transporters” or “drug efflux pumps.” Examples of drug efflux pumps include P-glycoprotein and multidrug resistance associated protein. This pumping of drugs causes multidrug resistance (MDR), a phenomenon that is observed in a variety of cancers. To improve the therapeutic effectiveness of a drug in a cell expressing drug efflux pumps (e.g., cancer cells), it is beneficial to use inhibitors of these pumps. Examples of multidrug transporter inhibitors include, without limitation, verampil, MC-207, 110(Phe-Arg-β-naphthylamide), 5′-methoxyhydnocarpin, INF 240, INF 271, INF 277, INF 392, INF 55, reserpine, GG918, diterpene from lycopus europaeus, epigallocatechin-3-O-gallate, progesterone, trifluoperazine, biricodar (VX-710), XR9576, tariquidar (XR9576), ceraminde, protein kinase C inhibitor (H7), n-methylwelwitindolinone C, isothiocyanate (welwistatin), cyclosoporin A, erythromycin, quinine, fluphenazine, tamoxifen, cremphor EL, dexverapamil, dexniguldipine, vaispodar, tariquidar, zosuquidar, laniquidar, elacridar, GF120918, novobiocin, fumitremorgin C, BIB-E, favopiridol, CI1033, iressa, VX-853, diethylstilbestrol, estrone, antiestrogens, TAG-11, TAG-139, toremifene, ONT-093, R-101933, mitotane, OC-144-093, LY-335979, annamycin, XR-9576, R-101933, and dofediquar (MS-209). In certain embodiments, a subject in need thereof is treated with a mucin inhibitor and/or a mucolytic agent and at least one of a multidrug transporter inhibitor, an N-glycosylation inhibitor, and a sialyltransferase inhibitor, prior to, or at substantially the same time as, treatment with a therapeutic agent.

The mucin inhibitor, mucolytic agent, multidrug transporter inhibitor, N-glycosylation inhibitor, sialyltransferase inhibitor, and therapeutic agent (collectively “agents”) may be administered to a mammal by a wide variety of routes, including enteral, parenteral, and topical. For example, the agents may be administered orally, intranasally, by inhalation, intramuscularly, subcutaneously, intraperitonealy, intravascularly, intravenously, transdermally, subcutaneously, or any combination thereof. In one instance, in the context of lung cancer or thyroid cancer, the agents can be administered intranasally (e.g., via an aerosol spray). In another instance, in the context of pancreatic cancer, prostate cancer, colon cancer, breast cancer, and ovarian cancer, the agents can be administered directly into the tumor. In certain situations, the agents are administered via a sustained release device.

The particular dose of the agents administered according to these aspects of the invention will, of course, be determined by the particular circumstances surrounding the case, including the agent administered, the particular mucinous carcinoma being treated, and the condition of the subject. Each of the agents are administered to a mammal in a therapeutically-effective amount. Such an amount is effective in treating or reducing symptoms of the mucinous carcinoma. This amount may vary, depending on the activity of the agent utilized, whether any other agent is co-administered, and the nature of such agent, the nature of the mucinous carcinoma, and the health of the patient. Although such amounts may be determined by the skilled artisan, typical therapeutically-effective amounts of mucin inhibitors include about 0.001 mg/kg/day to about 100 mg/kg/day; about 0.01 mg/kg/day to about 100 mg/kg/day; about 0.1 mg/kg/day to about 100 mg/kg/day; about 1 mg/kg/day to about 50 mg/kg/day; about 5 mg/kg/day to about 50 mg/kg/day; and about 10 mg/kg/day to about 200 mg/kg/day. In the case of mucolytic agents, the dosage is typically between about 50 mg/day to about 750 mg/day. The mucolytic agents may be administered once to about six times daily. Administration may be as a tablet, a liquid solution or in a manner suitable for inhalation. For example, erdosteine is typically administered at a dosage of about 300 mg twice daily; acetylcysteine is typically administered at a dosage of about 200 mg to about 300 mg twice daily; broheskin is administered at a dosage of about 5 to about 25 mg four times daily; carbocysteine is administered at a dosage of about 300 mg to about 500 mg daily; and guiafenesin is generally administered at a dosage of about 100 mg to about 500 mg daily. Of course, lower or higher dosages may be needed depending on the specific case. When the agents are combined with a carrier, they may be present in an amount of about 1 weight percent to about 99 weight percent, the remainder being composed of a pharmaceutically-acceptable carrier.

The agents may be administered in a pharmaceutically-acceptable carrier. Pharmaceutically-acceptable carriers and their formulations are well-known and generally described in, for example, Remington: The Science and Practice of Pharmacy (20th Edition, ed. A. Gennaro (ed.), Lippincott, Williams & Wilkins, 2000). In some embodiments, the pharmaceutically-acceptable carrier is in the form of an aerosol. Any suitable, pharmaceutically-acceptable carrier known in the art may be used. Carriers may be solid, liquid, or a mixture of a solid and a liquid. When present as a liquid or a mixture of a solid and a liquid, carriers that efficiently solubilize the agents are preferred. The carriers may take the form of capsules, tablets, pills, powders, lozenges, suspensions, emulsions or syrups, or other known forms. The carriers may include substances that act as flavoring agents, lubricants, solubilizers, suspending agents, binders, stabilizers, tablet disintegrating agents, and encapsulating materials. Solid or liquid carriers may be taken in the form of an aerosol to deliver the agents to their desired location, such as when used in a nebulizer for inhaling the agent.

Tablets for systemic oral administration may include excipients, as known in the art, such as calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with disintegrating agents, such as maize, starch or alginic acid, binding agents, such as gelatin, collagen or acacia and lubricating agents, such as magnesium stearate, stearic acid or talc. In powders, the carrier is a finely-divided solid which is mixed with an effective amount of a finely-divided mucin inhibitor and/or mucolytic agent. In solutions, suspensions or syrups, an effective amount of the mucin inhibitor and/or mucolytic agent is dissolved or suspended in a carrier such as sterile water, saline, or an organic solvent, such as aqueous propylene glycol. Other compositions can be made by dispersing the mucin inhibitor and/or mucolytic agent in an aqueous starch or sodium carboxymethyl cellulose solution or a suitable oil known to the art.

### Compositions and Kits

Aspects of the invention provide compositions comprising a mucin inhibitor and/or a mucolytic agent and at least one of a multidrug transporter inhibitor, an N-glycosylation inhibitor, and a sialyltransferase inhibitor. In some cases, these components are co-formulated. In other cases, they are provided as separate components that can be administered in any manner decided upon by a clinician. The mucin inhibitor, mucolytic agent may also be co-formulated with a therapeutic agent if necessary.

The compositions can be prepared in dosage unit form for ease of administration and uniformity of dosage. “Dosage unit form” as used herein, refers to physically-discrete units suited as unitary dosages for the subject to be treated; each unit containing a predetermined quantity of active compound calculated to produce the desired therapeutic effect in association with the selected pharmaceutical carrier.

In the case of an inhalation method, such as metered dose inhaler, the device is designed to deliver an appropriate amount of a formulation. For administration by inhalation, the compounds are delivered in the form of an aerosol spray from a pressured container or dispenser that contains a suitable propellant (e.g., a gas), such as carbon dioxide, or a nebulizer. Alternatively, an inhaled dosage form may be provided as a dry powder using a dry powder inhaler.

The compositions described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences, 20th edition (supra).

Sustained-release preparations of the compositions described herein can also be prepared. Suitable examples of sustained-release preparations include semi-permeable matrices of solid hydrophobic polymers containing the protein formulation. Examples of sustained-release matrices include polyesters, hydrogels (for example, poly(2-hydroxyethyl-methacrylate), or poly(vinylalcohol)), polylactides, copolymers of L-glutamic acid and γ-ethyl-L-glutamate, non-degradable ethylene-vinyl acetate, degradable lactic acid-glycolic acid copolymers, and poly-D-(−)-3-hydroxybutyric acid. The sustained-release formulations of the agents described herein can be developed using, e.g., polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years, depending on its molecular weight and composition. Liposomal compositions can also be used to formulate the compositions disclosed herein.

In another aspect of the invention, an article of manufacture or device is provided that contains at least one of a mucin inhibitor, a mucolytic agent, a multidrug transporter inhibitor, an N-glycosylation inhibitor, a sialyltransferase inhibitor, and a therapeutic agent, and typically provides instructions for its use. The device comprises a container suitable for containing the substances described above. Suitable containers include, without limitation, bottles, vials (e.g., dual chamber vials), syringes (e.g., single or dual chamber syringes), test tubes, nebulizers, inhalers (e.g., metered dose inhalers or dry powder inhalers), or depots. The container can be formed from a variety of materials, such as glass, metal, or plastic (e.g., polycarbonate, polystyrene, polypropylene). The container holds the substance and the label on, or associated with, the container can indicate directions for reconstitution and/or use. The label may further indicate that the substance is useful or intended for subcutaneous administration. The container holding the substance may be a multi-use vial, which allows for repeat administrations (e.g., from 2 to 6 doses) of the agent. The article of manufacture may further comprise a second container comprising a suitable diluent (e.g., WFI, 0.9% NaCl, BWFI, or phosphate buffered saline). The article of manufacture may further include other materials desirable from a commercial and user standpoint, including other buffers, diluents, filters, needles, syringes, and package inserts with instructions for use.

The invention will be more fully understood by reference to the following examples. They should not, however, be construed as limiting the scope of the invention.

### EXAMPLES

### Example 1

### Relative Sensitivity and Selectivity of MUC1 (CD227) Antibody for Human Pancreatic Cancer Cells Assayed by Immunofluoresence

MUC1 is heterogeneously expressed in various physiological states. To determine the role of membrane-associated mucin during chemotherapy, the reliability of anti-MUC1 antibody (CD227) was first tested to report changes in O-glycosylation of peptide core in response to inhibition of O-glycosylation, or shedding of glycosylated functional groups. The interaction of CD227 was evaluated against two mucin-secreting human pancreatic cancer cell lines (Capan-1 and HPAF-II), and one (non mucin-secreting) human glioblastoma (U-87 MG) cell line as a negative control. Each cell line was incubated with FITC-conjugated CD227 and subsequently analyzed by DIC and fluorescence microscopy to establish sensitivity and selectivity levels of CD227. Specifically, sterile cover slips were placed in six-well plates (Corning, N.Y.). Cells were next seeded at 5×10⁵ per ml in the same 6-well plates. Following an incubation period of 24 hr at 37° C., 4 μl of fluorescein isothiocyanate (FITC)-conjugated mouse anti-human CD227 (MUC1) monoclonal antibody (BD Pharmingen, San Jose, Calif.) was added to each well. Cells were incubated for an additional 24 hr with antibody and washed with 1×PBS to remove unassociated antibody. The cover slip from each well was mounted onto a glass microslide (Corning, N.Y.) with fluor mounting media (Trevigen Inc, MD). The association of CD227 (anti-MUC1) antibody with cells was determined using a combination of fluorescence and DIC microscopic applications at 20× magnification (Olympus BX61WI, Melville, N.Y.).

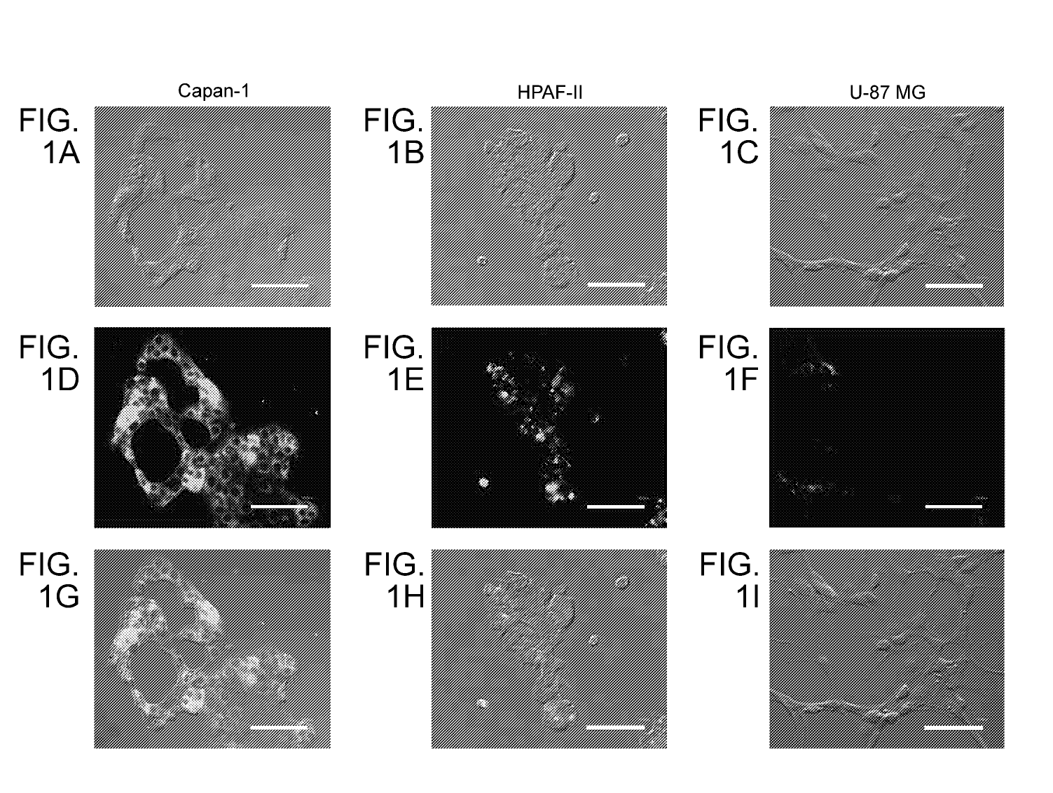

DIC images showed tightly joined clusters of Capan-1 and HPAF-II cells; images of areas surrounding individual cells were difficult to acquire (FIGS. 1A and 1B). FIG. 1C shows a uniform arrangement of U-87 MG cells. U-87 MG cells were more uniform in shape compared to Capan-1 and HPAF-II with limited intercellular associations and no cell clustering was observed.

Fluorescence images acquired for the interaction of CD227 with Capan-1 cells showed areas of intense antibody association, compared to relatively low uptake by HPAF-II and non-specific association with U-87 MG (FIGS. 1D-1F).

CD227 accurately distinguished between high and relatively low MUC1 mucin expression, and therefore represents a sensitive tool to evaluate the role of MUC1 during chemotherapy. Moreover, relative to Capan-1 and HPAF-II, U-87 MG failed to recognize CD227, and hence, strongly suggested that CD227 is a selective indicator of MUC1 positive expression (FIGS. 1G-GI). Moreover, the distribution of MUC1 was generally observed on the entire surface of human pancreatic cells, and our CD227 reactivity studies involving Capan-1 show MUC1 mucin on Capan-1 cell membranes surrounding the cytoplasmic and nuclear cell compartment (FIG. 1G).

### Example 2

### Determination of Maximum Non-Toxic Concentration of Benzyl-α-GalNAc for Removing Cell-Bound Mucin

O-glycosylation is critical for mucin formation. The elongation of O-glycosylated chains of the peptide core can be blocked by culturing MUC1 mucin secreting cells with benzyl-2-acetamido-2-deoxy-α-D-galactopyranoside (benzyl-α-GalNAc). In order to determine the contribution of MUC1 expression in the delivery of drugs such as 5-FU to pancreatic tumors, the maximum non-toxic concentration of benzyl-α-GalNAc that could be used to inhibit O-glycosylation of MUC1 was determined from cellular toxicity studies. These cellular toxicity studies were performed as follows. Cells were seeded at 1×10⁴ per ml in 48-well plates. Following a 24 hr incubation period at 37° C., cells were exposed to various concentrations of benzyl-α-GalNAc solution (Sigma-Aldrich, St. Louis, Mo.) prepared in media. After 72 hr of cell exposure to benzyl-α-GalNAc, the percent of cell viability was determined using sulforhodamine B assay (Kalra et al., Pharm. Res., 23(12):2809-2817 (2006); Skehan et al., J. Natl. Cancer Inst,. 82:1107-1112 (1990)) and calculated as follows:

$$Percent of cell viability = \frac{\begin{matrix}Flourescence intensity of \\ benzyl \text{-} α \text{-} GalNAc treated cells\end{matrix}}{\begin{matrix}Flourscence intensity of \\ non \text{-} treated cells ( control )\end{matrix}} \times 100$$

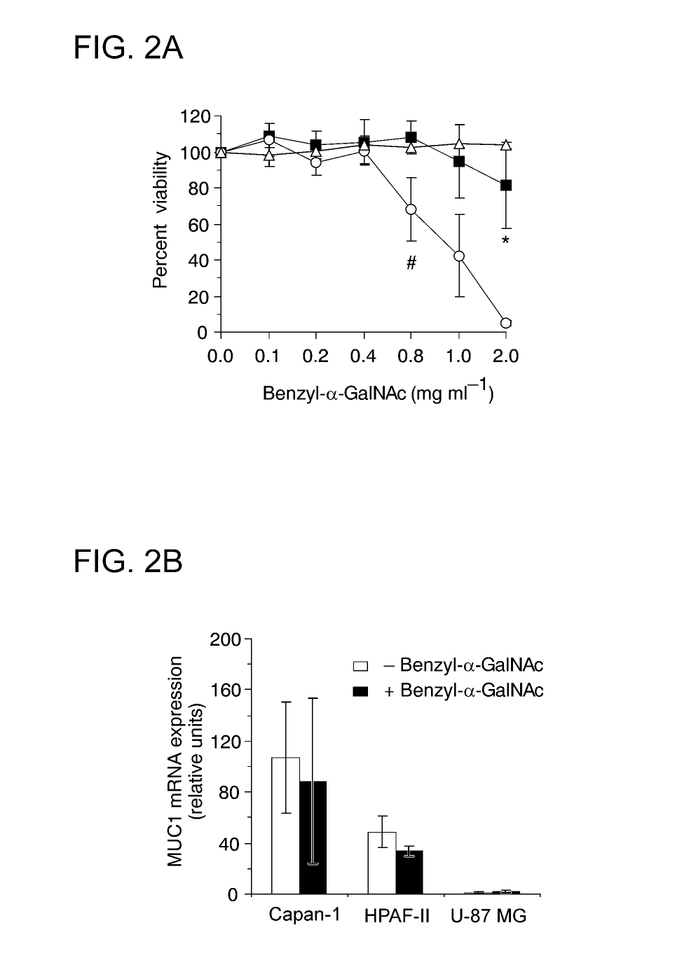

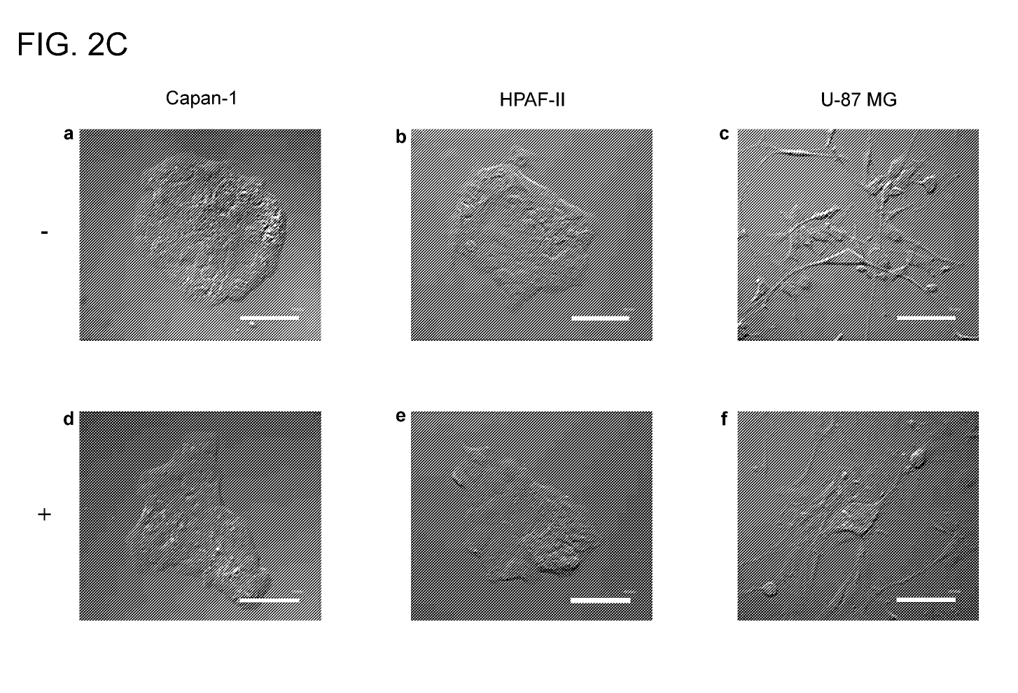

The maximum non-toxic concentration of benzyl-α-GalNAc for the pancreatic cancer cells Capan-1 and HPAF-II were 0.4 mg/ml and 0.8 mg/ml, respectively (FIG. 2A). The U-87 MG non-mucin secreting cell line tolerated >0.8 mg/ml.

To observe the effects of benzyl-α-GalNAc exposure on cell morphology, differential interference contract (DIC) microscopy was employed. Sterile cover slips were placed in six-well plates (Corning, N.Y.). Capan-1, HPAF-II, and U-87 MG cells were next seeded at 1×10⁴ per ml in the same 6-well plates. Following a 24 hr incubation period at 37° C., the cells were exposed to the maximum non-toxic concentration (determined above) of benzyl-α-GalNAc solution prepared in media. Following 72 hr of cell exposure to benzyl-α-GalNAc, each well was washed with 1×PBS and the cover slip from each well was mounted onto a glass microslide (Corning, N.Y.) with fluor mounting media (Trevigen Inc, MD). Finally changes in cell morphology were observed at 20× magnification (Olympus BX61WI, Melville, N.Y.). The morphological structures of cells after 72 hr of exposure to benzyl-α-GalNAc showed no visible difference compared to cells not exposed to the inhibitor (FIG. 2B). Thus, the cell lines varied in their ability to tolerate general effects of the benzyl-α-GalNAc inhibitor, and the maximum non-toxic concentrations vary and do not correlate with the cells' ability to secrete mucin.

### Example 3

### Confirming the Inhibition of MUC1 O-Glycosylation by Determination of Alterations in Anti-MUC1 Antibody Association

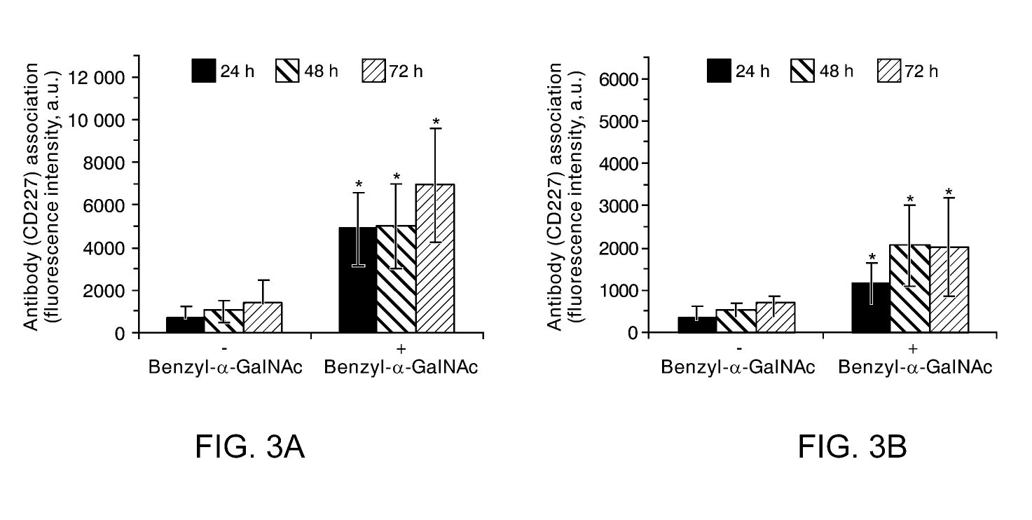

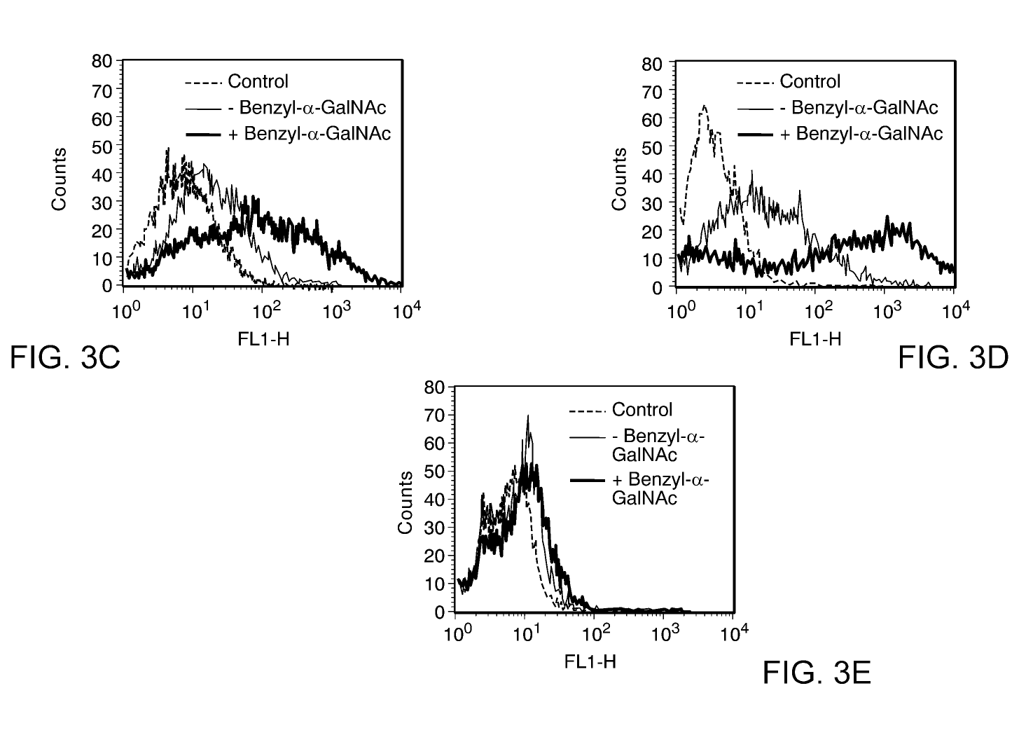

In order to determine whether the concentrations of benzyl-α-GalNAc employed are sufficient to inhibit MUC1 O-glycosylation, we observed the alterations in anti-MUC1 (CD227) association after exposure to the inhibitor. The antibody association studies were performed as follows. Cells were seeded at 2×10⁴ per ml in 24-well plates. Following a 24 hr incubation period at 37° C., cells were exposed to the maximum non-toxic concentration of benzyl-α-GalNAc for 24 hr, 48 hr, and 72 hr. After each exposure time point the cells were washed with 1×PBS and 1 ml of fresh media was added to each well. The cells were then exposed to 4 μl/well of anti-MUC1 antibody. Following 24 hr incubation with the antibody, cells were washed with 1×PBS to remove any unassociated antibody and the fluorescence intensity was measured using a fluorescence microplate reader (Bio-Tek® Instruments Inc., VT) at excitation wavelength of 485 nm and emission wavelength of 528 nm. Antibody association with both Capan-1 (FIG. 3A) and HPAF-II (FIG. 3B) cell lines was significantly enhanced (*P 0.001) after 24 hr, 48 hr, or 72 hr of exposure to benzyl-α-GalNAc (+) as compared to cells not exposed to benzyl-α-GalNAc (−).

Fluorescence-activated cell-sorting (FACS) analysis was then performed as follows. Cells were seeded at 2×10⁴ per ml in 24-well plates. Following a 24 hr incubation period at 37° C., cells were exposed to the maximum non-toxic concentration of benzyl-α-GalNAc. After 48 hr exposure to benzyl-α-GalNAc cells were washed with 1×PBS and 1 ml of fresh media was added to each well. The cells were then exposed to 4 μl/well of CD227 antibody. Following 24 hr incubation with the antibody, cells were washed with 1×PBS to remove any unassociated antibody and cells were detached using 0.5 ml/well trypsin. Cells were then washed with 1×PBS and antibody association was determined using FACS analysis. The FACS analysis data revealed a shift in fluorescence peaks for cells exposed to benzyl-α-GalNAc as compared to cells not exposed to the inhibitor (FIG. 3C and FIG. 3D), whereas no shift in peak was seen for the negative control U-87 MG cells (FIG. 3E). These results collectively suggest that the non-toxic concentrations of benzyl-α-GalNAc used in the study were capable of inhibiting the MUC1 O-glycosylation thereby enhancing the accessibility of CD227 antibody to the MUC1 peptide recognition site.

### Example 4

### Mucin is a Cellular Barrier Limiting Chemotherapeutic Action of 5-FU Against Human Pancreatic Cancer Cells

Given the rapid turnover of mucin in adenocarcinomas, these experiments were conducted to determine whether the inhibition of O-glycosylation will occur only while cells are exposed to the effect of the inhibitor.

The effects of increasing benzyl-α-GalNAc concentrations on cytotoxicity of 5-FU (108 μmol/ml) against HPAF-II cells was first tested. Cells were seeded at 1×10⁴ per ml in 48-well plates. Following a 24 hr incubation period at 37° C., one row of cells was exposed to non-toxic concentration of benzyl-α-GalNAc solution prepared in media, whereas another row of cells was allowed to grow in media without benzyl-α-GalNAc. Following 48 hr of exposure cells in each group were washed with 1×PBS and treated with 5-FU solution (Sigma-Aldrich, St. Louis, Mo.) prepared in media. The growth inhibitory effect of 5-FU after 24 hr of exposure was determined using sulforhodamine B (Sigma-Aldrich, St. Louis, Mo.) assay the percent of viable cells was calculated as follows:

$$Percent of cell viability = \frac{\begin{matrix}Fluorescence intensity of cells \\ treated with drug ( 5 \text{-} FU )\end{matrix} }{\begin{matrix}Fluorescence intensity of \\ non \text{-} treated cells ( control )\end{matrix} } \times 1$$

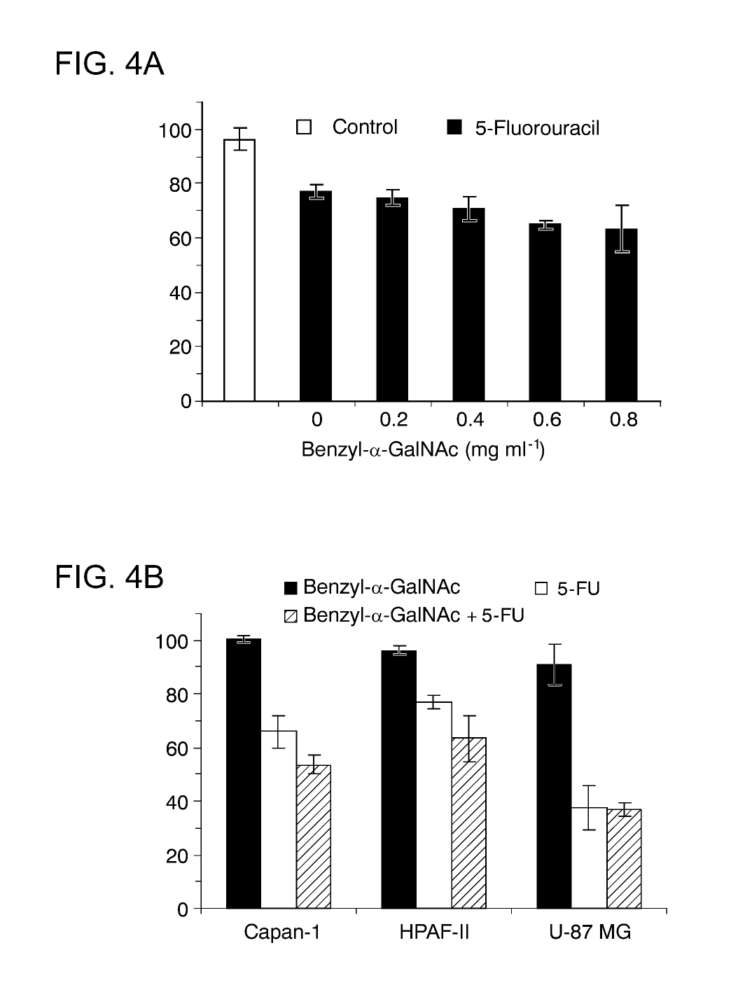

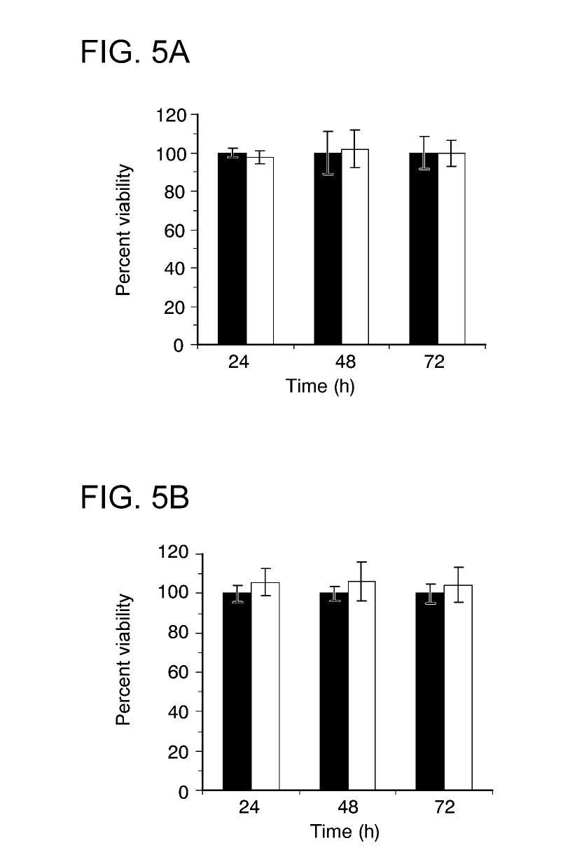

The percent cell viability was found to decrease with increasing concentrations of the inhibitor, and this effect was significant (*P≦0.001) at concentrations≧0.4 mg/ml as compared to 5-FU treatment alone (FIG. 4A). The percent viability with 5-FU treatment alone was 77.1±2.5% which was significantly lowered to 63.4±8.6% when pretreated with the maximum non-toxic concentration of benzyl-α-GalNAc (0.8 mg/ml). Similar results were observed for Capan-1 cells wherein the percent viability was 65.9±2% with 5-FU (80 μmol/ml) alone, which was significantly (*P 0.001) lowered to 53.6±3.6% when exposed to benzyl-α-GalNAc (0.4 mg/ml) followed by 5-FU treatment (FIG. 4B). There was no significant difference in percent cell viability of our control U-87 MG cells which remained the same at 37% when cells were exposed to 5-FU alone or to benzyl-α-GalNAc (0.8 mg/ml) prior to 5-FU treatment. These results suggest that the cytotoxic effect of 5-FU against MUC1 secreting cell lines was enhanced by inhibition of O-glycosylation.

### Example 5

### Effect of Removing Membrane-Associated Mucin

### Time-Dependent Analysis

These experiments were conducted to determine whether the successful elimination of the glycosylated functional groups surrounding the MUC1 peptide core limit the capacity of cells to proliferate once benzyl-α-GalNAc has been removed. A significant change in cells' ability to multiply and divide due to exposure to benzyl-α-GalNAc would affect the measure of MUC1's role in our experiment. Capan-1 and HPAF-II cells were exposed to benzyl-α-GalNAc for 48 hr. Percent of cell viability was determined 24 hr, 48 hr, and 72 hr after removal of benzyl-α-GalNAc. Specifically, cells were seeded at 1×10⁴ per ml in 48-well plates. Following a 24 hr incubation period at 37° C., cells were exposed to the maximum non-toxic concentration of benzyl-α-GalNAc solution prepared in media. Following 48 hr exposure to benzyl-α-GalNAc cells were washed with 1×PBS and then allowed to grow for an additional 24 hr, 48 hr, and 72 hr in fresh media. After each time point post benzyl-α-GalNAc exposure, the percent of cell viability was determined using sulforhodamine B assay and morphological changes were observed using DIC microscopy.

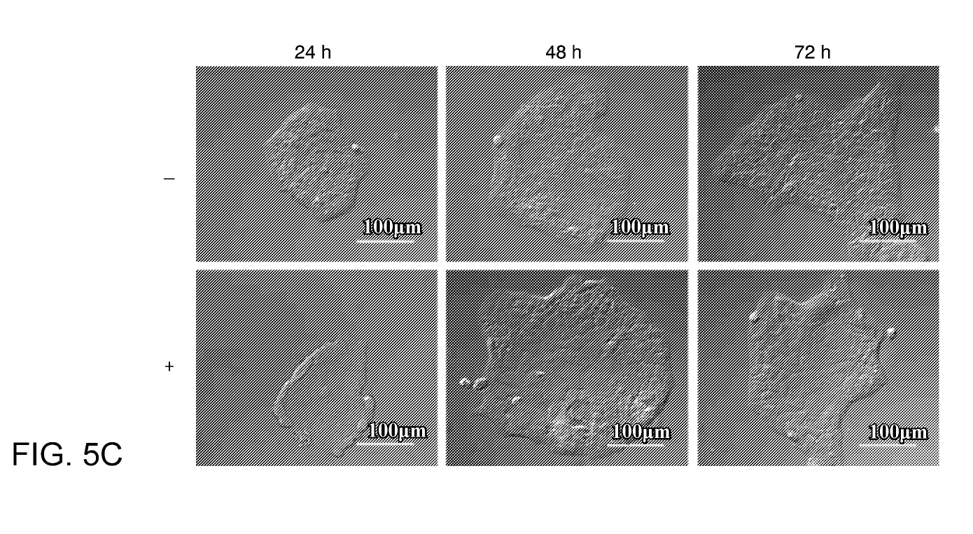

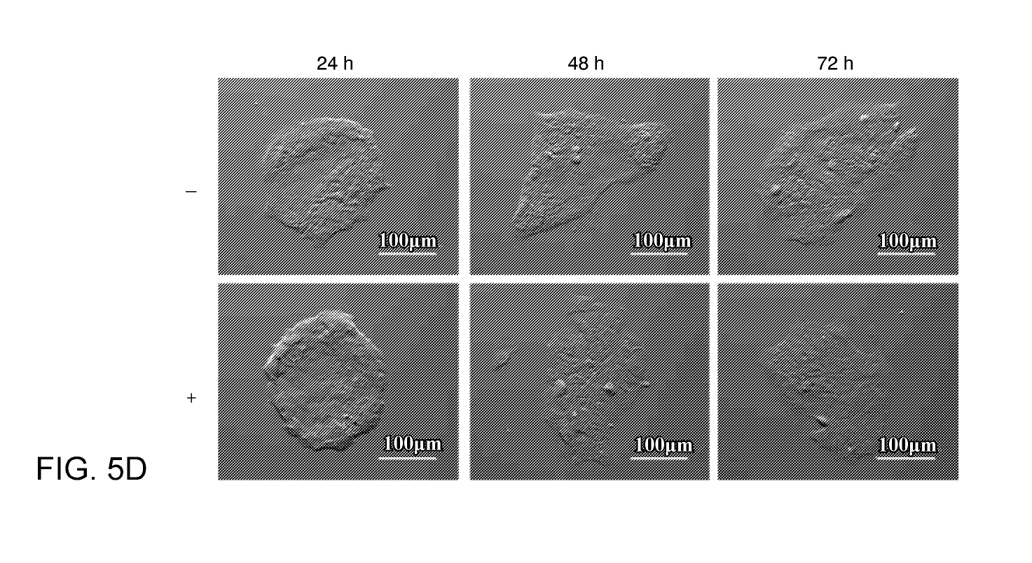

No significant effect of inhibitor on growth of Capan-1 (FIG. 5A) and HPAF-II (FIG. 5B) cells post-removal of benzyl-α-GalNAc, was observed compared to untreated controls. Further, DIC images of cells post-removal of benzyl-α-GalNAc showed no visible morphological changes in cellular structures for both cell lines (FIG. 5C and FIG. 5D).

These data support the hypothesis that the concentration of benzyl-α-GalNAc selected for pre-treatment of Capan-1 and HPAF-II cells was non-toxic for several days post-removal of the inhibitor. Under these conditions the effect of chemotherapeutic agents (i.e., 5-FU) against cells previously exposed to benzyl-α-GalNAc was due to the chemotherapeutic agent and not exposure to benzyl-α-GalNAc.

### Example 6

### Effects of Removing Sialic Acid Residues on 5-FU Cytotoxicity

In several pancreatic cancer cells, the presence of terminal sialic acid sugar moiety on carbohydrate chains is higher due to the overexpression of sialyltransferases (Peracula et al., Glycoconj. J., 22:135-144 [2005]). These experiments were performed to determine whether removing the terminal sialic acid groups alone would enhance the cytotoxic effects of 5-FU, or if it is essential to inhibit the entire carbohydrate chain formation using benzyl-α-GalNAc.

The enzyme neuraminidase, which cleaves terminal sialic acid residues, was used in these studies (Varki et al., J. Biol. Chem., 258:12465-12471 [1983]). Cells were seeded at 1×10⁴ per ml in 48-well plates. Following 24 hr incubation period at 37° C., cells were exposed to 0.05 U/ml for 1 hr. The removal of sialic acid residues from cell surface was confirmed by labeling cells with 5 μl/well FITC conjugated MAA lectin for 30 mins. The effects of neuraminidase on CD227 antibody association was determined by labeling cells with 5 μl/well CD227 antibody for 4 hr. The fluorescence intensities were measured using a fluorescence microplate reader (Bio-Tek® Instruments Inc., VT) at excitation wavelength of 485 nm and emission wavelength of 528 nm. The effects of neuraminidase on 5-FU cytotoxicity was determined by exposing cells to 0.05 U/ml neuraminidase for 1 hr followed by treatment with 5-FU for 24 hr. Percent cell viability was determined using sulforhodamine B assay.

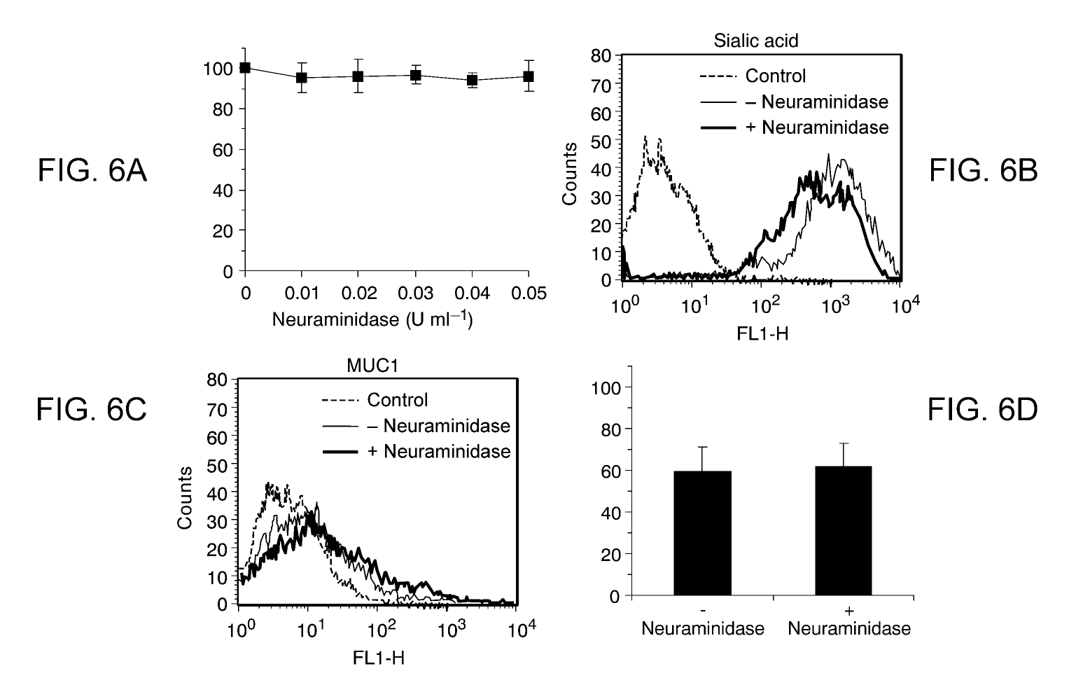

Neuraminidase concentrations up to 0.05 U/ml had no toxic effects on HPAF-II cells (FIG. 6A). HPAF-II cells were exposed to 0.05 U/ml of neuraminidase and the removal of sialic acid groups was confirmed using FITC conjugated MAA lectin, which binds to sialic acid residues (Wang et al., J. Biol. Chem., 263:4576-4585 [1988]). The significant decrease in fluorescence intensities for cells exposed to neuraminidase confirmed the removal of sialic acid groups (FIG. 6B). The effect of sialic acid removal on association of CD227 antibody showed no significant change in antibody association between cells exposed to neuraminidase and control cells (FIG. 6B). The cytotoxic effects of 5-FU was then determined against the cells exposed to neuraminidase. The percent viability of cells treated with 5-FU alone was 49%±4.6%, which was not significantly altered when cells were exposed to neuraminidase prior to 5-FU treatment (FIG. 6C).

### Example 7

### Inhibition of MUC1 O-Glycosylation in Pancreatic Tumors

As described above, the two human pancreatic cancer cell lines, Capan-1 and HPAF-II, showed relatively high and moderate levels of MUC1, respectively, whereas the U-87 MG cells showed no MUC1 expression. In this experiment, the levels of MUC1 expression within tumor xenografts obtained from these cell lines was determined.

All animal work was performed in the animal facility at Northeastern University, Boston, Mass. in accordance with the institutional guidelines. Female SCID (severe combined immunodeficient mice), 6 to 8 weeks old purchased from Massachusetts General Hospital, Boston, Mass. were used for our study since they have been used successfully for developing human xenografts of pancreatic tumors. To establish the tumors, 2.5×10⁶ Capan-I cells in 0.1 ml of cell culture medium were injected into the subcutaneous dorsa of all mice. Mice were weighed daily using a digital weighing balance (AccuSeries®, Fisher Scientific, Arvada, Colo.) and tumor volumes were also measured every day using an electronic digital caliper (Control Company, Friendswood, Tex.). Tumor volumes were calculated using the formula a²×b×0.52, where “a” is the longer diameter and “b” is the shorter diameter. Once the tumor volumes were approximately 50 mm³-70 mm³ the experimental groups received intratumoral injections of benzyl-α-GalNAc (0.1 ml injection; 10 mg/ml) whereas the control groups were given at intervals of 48 hr. When the tumor volumes were approximately 100 mm³ 5-FU was administered via the intravenous route. Animals in treatment groups received two injections (0.1 ml) of 5-FU at interval of 4 days whereas control mice received saline (0.1 ml). The total dose of 5-FU administered was 125 mg/kg. At the end of the experiment animals were sacrificed, tumor tissue was surgically removed and fixed in 10% formalin at 4° C. for histochemical staining.

For histochemical staining, paraffin-embedded tissue sections (5 μm) were stained with hematoxylin and eosin (H&E) to evaluate and compare the extent of tissue viability. The tumor sections were scanned and qualitative images captured using brightfield microscopy (Olympus BX61WI, NY). The number of tumor cells in each section was quantified using bioquant imaging software and expressed as percent of total tumor area. To quantify the total tumor area we first traced the outline of the entire tumor section in each field of view (FOV). The pixels within the traced area were then selected and the pixel count was determined by the software. A total of five FOV were analyzed to determine the total pixel count of the tumor area. Microscope stage encoders were used to ensure that no tumor area was counted more than once. Similarly the total pixel count of the neoplastic cells featured in blue in each FOV was determined. The total percent of neoplastic cell density was calculated as follows:

$$Percent cell density = \frac{Total pixel count for cells ( blue ) stained}{Total pixel count for entire tumor area} \times 100$$

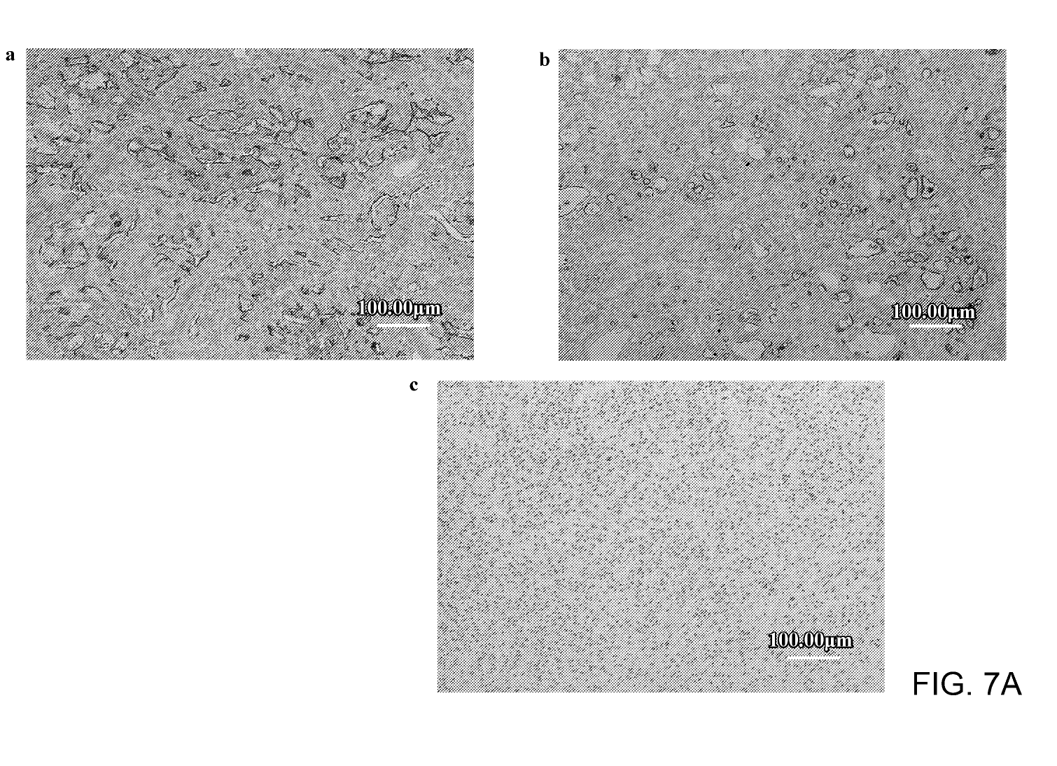

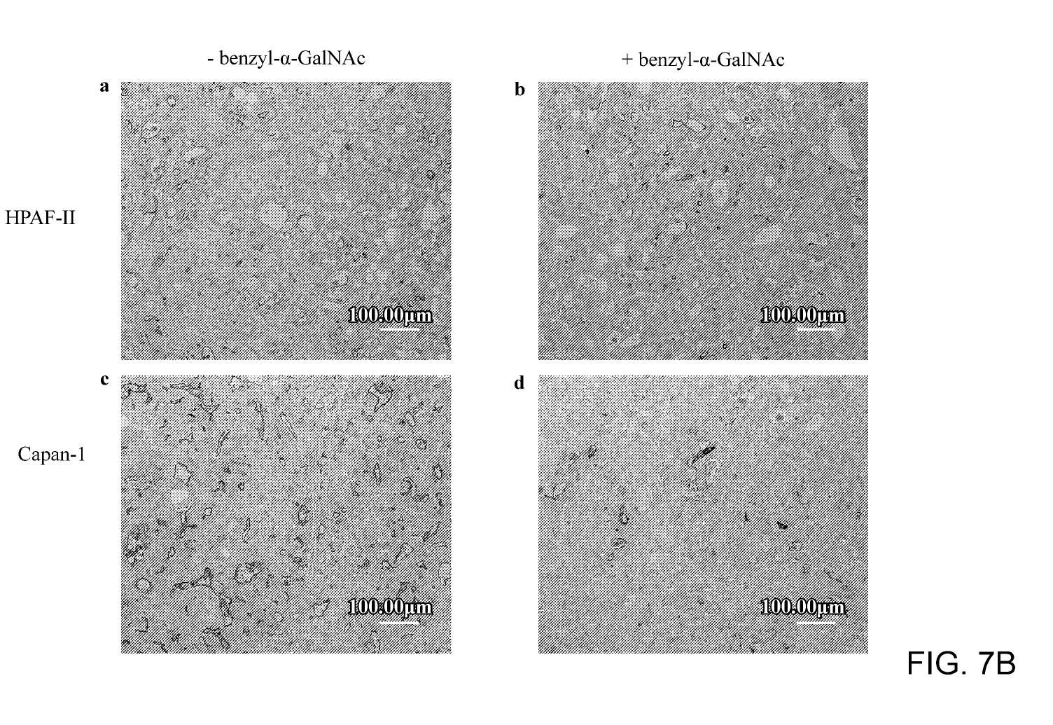

Immunohistochemical staining of tumor section was performed with anti-MUC1 antibody (FIG. 7A). Capan-1 tumors (FIG. 7A) showed high MUC1 staining as compared to moderate staining in HPAF-II tumors (FIG. 7A to FIG. 7B), whereas U-87 MG tumors showed no staining with anti-MUC1 antibody (FIG. 7A to FIG. 7C).

In order to test the in vivo effect of Benzyl-α-GalNAc in a pancreatic tumor model, Benzyl-α-GalNAc was administered locally into the tumor mass of mice via intratumoral injections (10 mg/ml, 0.1 cc). A total of four intratumoral injections were given at intervals of 48 hr. Following intratumoral injections, the sections of subcutaneous Capan-1 and HPAF-II tumors were analyzed for MUC1 carbohydrate staining (FIG. 7B). Lower staining for MUC1 O-glycosylation was observed in tumors exposed to benzyl-α-GalNAc for both HPAF-II (FIG. 7B) and Capan-1 (FIG. 7B to FIG. 7D) tumors as compared to tumors not exposed to the inhibitor (FIG. 7Ba and FIG. 7Bc).

These results show that intratumoral injections of benzyl-α-GalNAc were capable of inhibiting MUC1 mucin content in pancreatic tumors.

### Example 8

### Effects of Inhibiting Mucin O-Glycosylation on Anti-Tumoral Activity of 5-FU

This experiment was performed to determine whether inhibition of mucin O-glycosylation could enhance the ability of pancreatic cancer cells to respond to 5-FU in vivo.

For these studies the pancreatic cell line, Capan-1 was used to used to establish a pancreatic tumor model. Capan-1 tumors were established in subcutaneous dorsa of female SCID mice (see, Example 7). When the tumor size reached approximately 50 mm³ to 70 mm³, intratumoral injections of benzyl-α-GalNAc (0.1 ml, 10 mg/ml) were administered on days 4, 6, 8, and 10, while the control groups received comparable injections of saline. 5-FU therapy began when the tumor size was approximately 100 mm³.

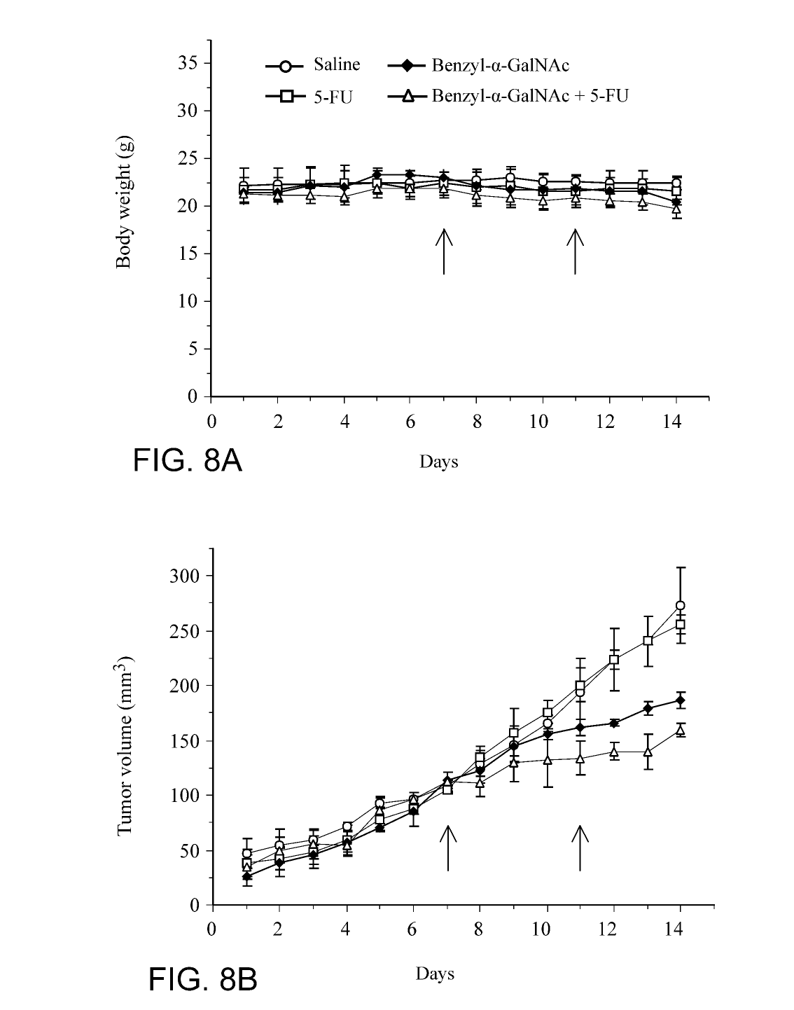

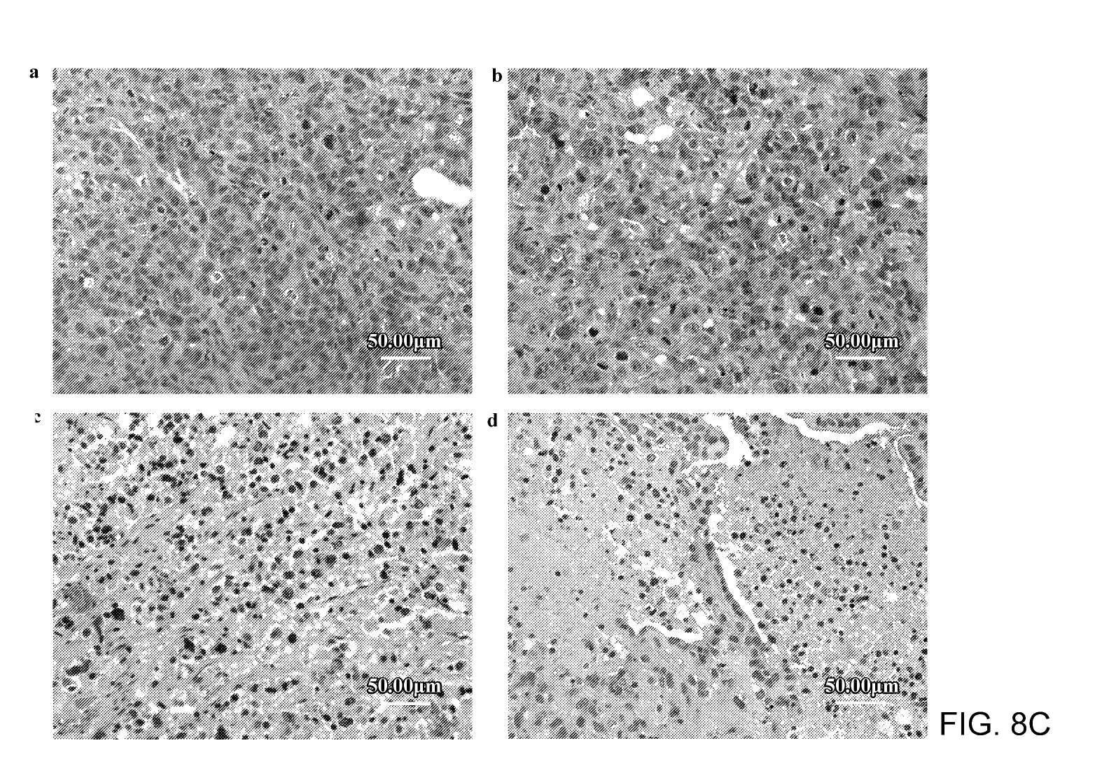

FIG. 8A shows the body weight of animals monitored during the course of the experiment. There were no significant changes observed in weight of animals in all experimental groups compared to controls. This data suggests that the treatment regime was well tolerated.

The tumor volume was monitored as an indicator of response to 5-FU treatment (FIG. 8B). No difference was observed between the tumor volumes in control animals receiving intratumoral injections of saline when compared to injections of benzyl-α-GalNAc, suggesting that the intratumoral injections of the mucin inhibitor did not alter the rate of tumor growth. The groups that received 5-FU treatment (either 5-FU alone of benzyl-α-GalNAc+5-FU) had significantly lower (p=0.05) tumor volumes as compared to control tumors (saline group), suggesting that the dose of 5-FU was able to suppress tumor growth. The tumors pre-treated with mucin O-glycosylation inhibitor followed by 5-FU treatment (benzyl-α-GalNAc+5-FU group) showed significantly lower tumor volumes (p=0.05) as compared to 5-FU treatment alone (intratumoral saline+5-FU).

The histochemical staining of tumor sections showed dense packing of neoplastic cells in both the control groups, receiving either intratumoral injections of saline (FIG. 8Ca) or benzyl-α-GalNAc (FIG. 8Cb). These images suggest that intratumoral injections of the mucin inhibitor did not alter general cell and tissue matrix organization within the tumors. The tumors that received 5-FU treatment (either 5-FU alone (FIG. 8Cc) or benzyl-α-GalNAc+5-FU (FIG. 8Cd) showed fewer tumor cells compared to the control tumors. The tumor cells within these sections were found to be more dispersed throughout the tissue matrix. The tumors treated with benzyl-α-GalNAc+5-FU showed lower numbers of neoplastic cells when compared with those treated with 5-FU alone (FIG. 8Cc and FIG. 8Cd).

The neoplastic cell densities within the tumor sections as a percent of total tumor area was quantified using bioquant imaging software (Table 1).

TABLE 1

| Group # | Treatment | Percent Tumor Area |
|---|---|---|
| 1 | Saline | 27.9 ± 9.4 |
| 2 | Benzyl-α-GalNAc | 28.3 ± 1.8 |
| 3 | 5-FU | 21.0 ± 1.4 |
| 4 | Benzyl-α-GalNAc + 5-FU | 11.4 ± 5.6 |

The control groups receiving either intratumoral injections of saline or benzyl-α-GalNAc showed no difference in total cell densities. The tumors treated with 5-FU showed significantly lower (p=0.05) tumor cell densities when compared to control groups. The neoplastic cell density within the tumors pre-treated with benzyl-α-GalNAc followed by 5-FU was significantly lower (p=0.05) when compared to 5-FU treatment alone. These results were consistent with the tumor volume data and collectively suggest that inhibition of mucin content within Capan-1 pancreatic tumors enhanced the anti-tumoral activity of 5-FU.

### Example 9

### Intracellular Uptake of 5-FU

The effectiveness of 5-FU treatment is a direct result of successful conversion of the 5-FU prodrug to its active metabolites (such as FUrd, FdUrd, FUTP and FdUMP) formed within the intracellular compartments of the cells. The total levels of metabolites formed within target cells largely depends upon the intracellular levels of 5-FU and the various enzymes participating in the conversion of the prodrug to active metabolites. The purpose of this experiment was to determine whether the enhanced therapeutic activity of 5-FU observed following the inhibition of mucin O-glycosylation was due to an increase in the uptake of 5-FU by these cells.

Capan-1 and U-87 MG cells were exposed to the maximum non-toxic concentration of benzyl-α-GalNAc for 48 hr followed by 1 hr and 4 hr exposure to 5-FU (50 μmol/ml). The intracellular levels of the drug were determined using a primary antibody against 5-FU followed by FITC-conjugated secondary antibody. Specifically, sterile cover slips were placed in 24-well plates (Corning, N.Y.). Cells were seeded at 2×10⁴ per ml in the same 24-well plates. Following a 24 hr incubation period at 37° C. cells was exposed to the maximum non-toxic concentration of benzyl-α-GalNAc solution prepared in cell culture media, the control cells were incubated in growth media without the O-glycosylation inhibitor. Following an additional 48 hr of incubation, each row was washed with 1×PBS and treated with 5-FU prepared in cell growth medium. The intracellular uptake of 5-FU following 1 hr and 4 hr of cell exposure to drug was determined by immunofluorescence labeling. Briefly, the medium containing 5-FU was aspirated and cells were washed twice with 1×PBS. The cells were then fixed with cold methanol for 10 min at −20° C. followed by rinsing with cold acetone. The cells were next washed twice with 1×PBS followed by 30 min rehydration in 1×PBS. For immunolabeling, cells were exposed to 200 μl (1:1000 dilution; stock concentration: 1 mg/ml) of primary antibody against 5-FU (Lampire Biological Laboratories, Pipersville, Pa.) for 1 hr at room temperature. The unassociated primary antibody was removed by washing three times with 1×PBS and cells were then incubated at room temperature with 200 μl (1:1000 dilution; stock concentration: 1 mg/ml) of fluorescein isothiocyanate (FITC)-conjugated goat anti-mouse IgG secondary antibody (Lampire Biological Laboratories, Pipersville, Pa.). Following 1 hr incubation with the secondary antibody, cells were washed twice with 1×PBS to remove any unassociated secondary antibody and the fluorescence intensity was measured using a fluorescence microplate reader (Bio-Tek® Instruments Inc., VT) at excitation wavelength of 485 nm and emission wavelength of 528 nm. The intracellular uptake of 5-FU was observed under a fluorescence microscope. The coverslip from each well was mounted onto a glass microslide (Corning, N.Y.) with SlowFade® Gold antifade reagent (Invitrogen, Carlsbad, Calif.). The images were captured using a combination of fluorescence and DIC microscopic applications at 20× magnification (Olympus BX61WI-Melville, N.Y.).

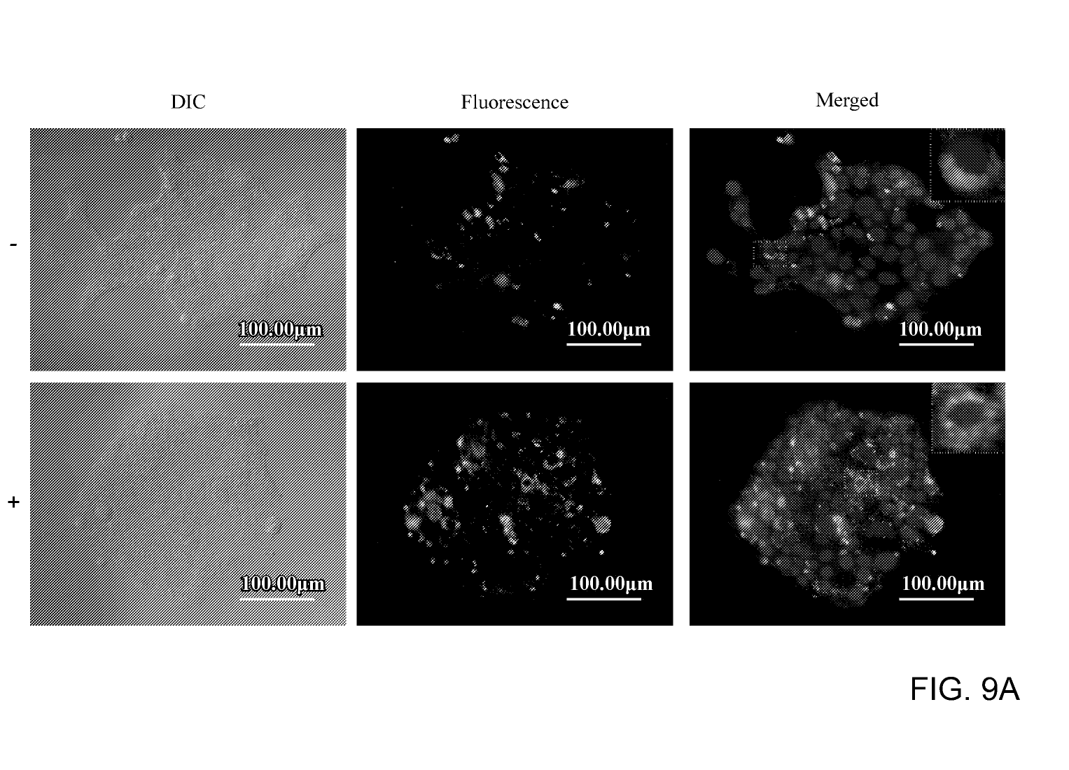

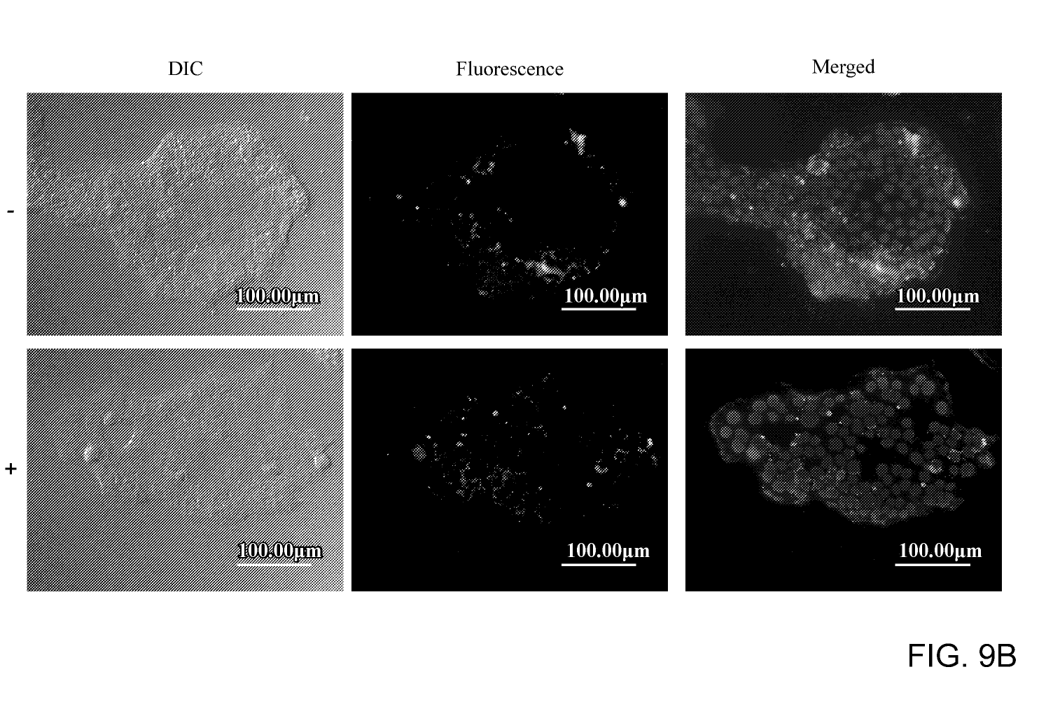

Fluorescent imaging studies showed higher fluorescence staining of intracellular 5-FU in Capan-1 cells pre-treated with benzyl-α-GalNAc followed by 1 hr of exposure to 5-FU as compared to cells treated with 5-FU alone (FIG. 9A). Intense cytoplasmic staining was observed surrounding the nucleus in Capan-1 cells pre-treated (+) with the inhibitor (FIG. 9A:inset) as compared to untreated controls (−). The fluorescent images observed for Capan-1 cells following 4 hr of 5-FU exposure (FIG. 9B) showed no difference in qualitative antibody uptake between cells pre-treated (+) with O-glycosylation inhibitor compared to controls (−).

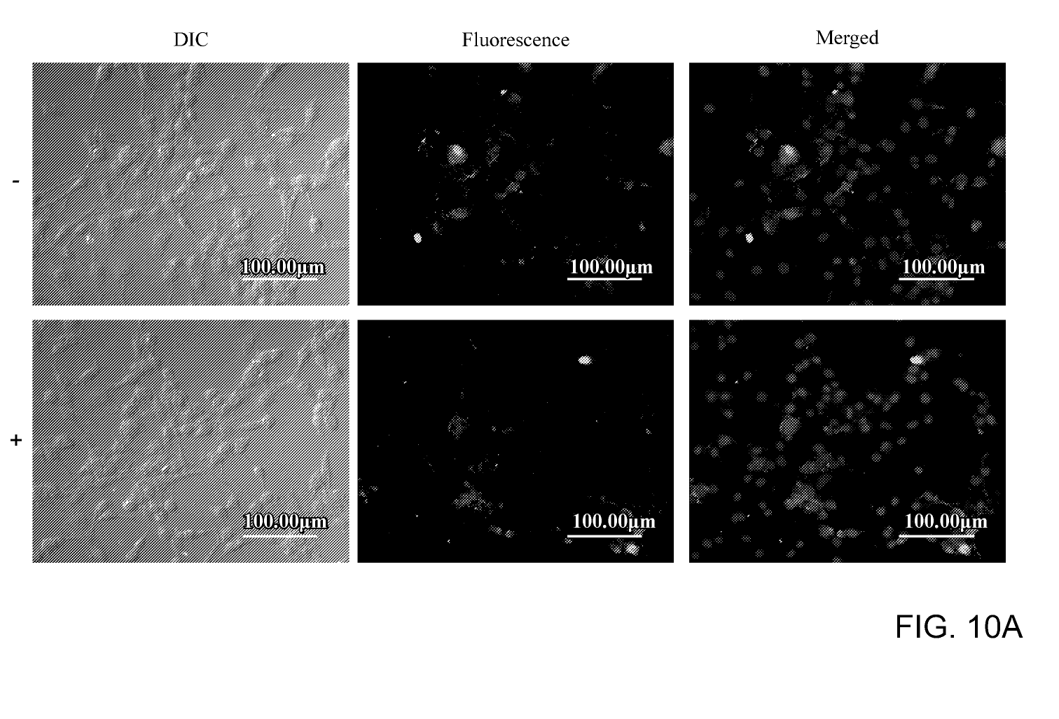

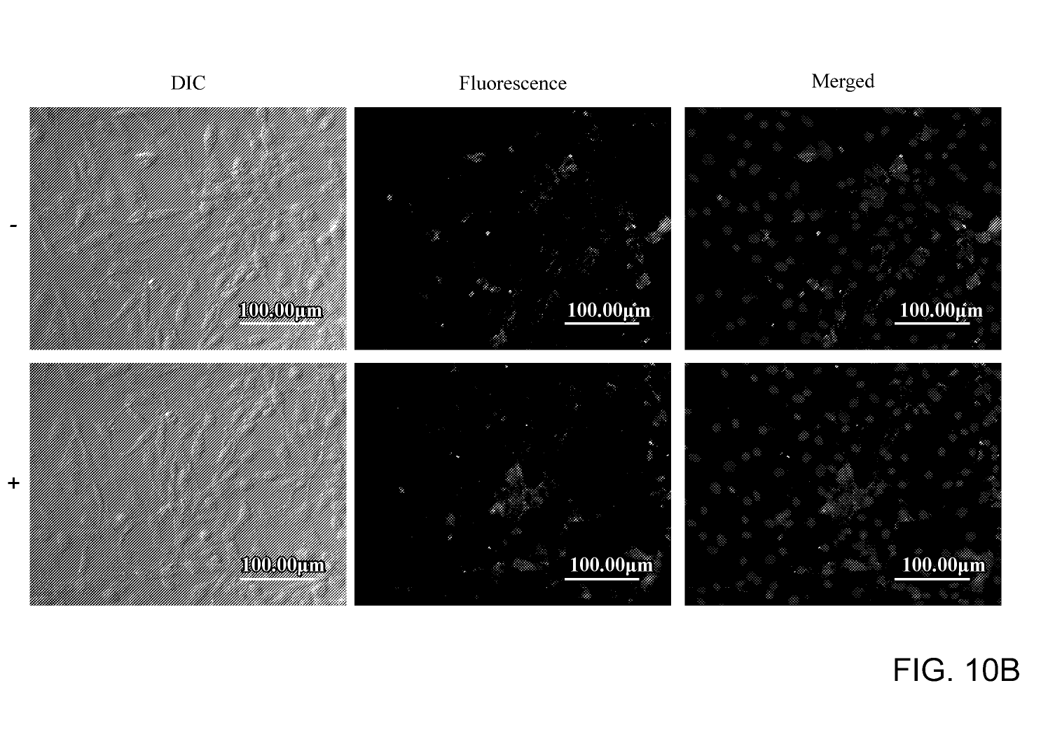

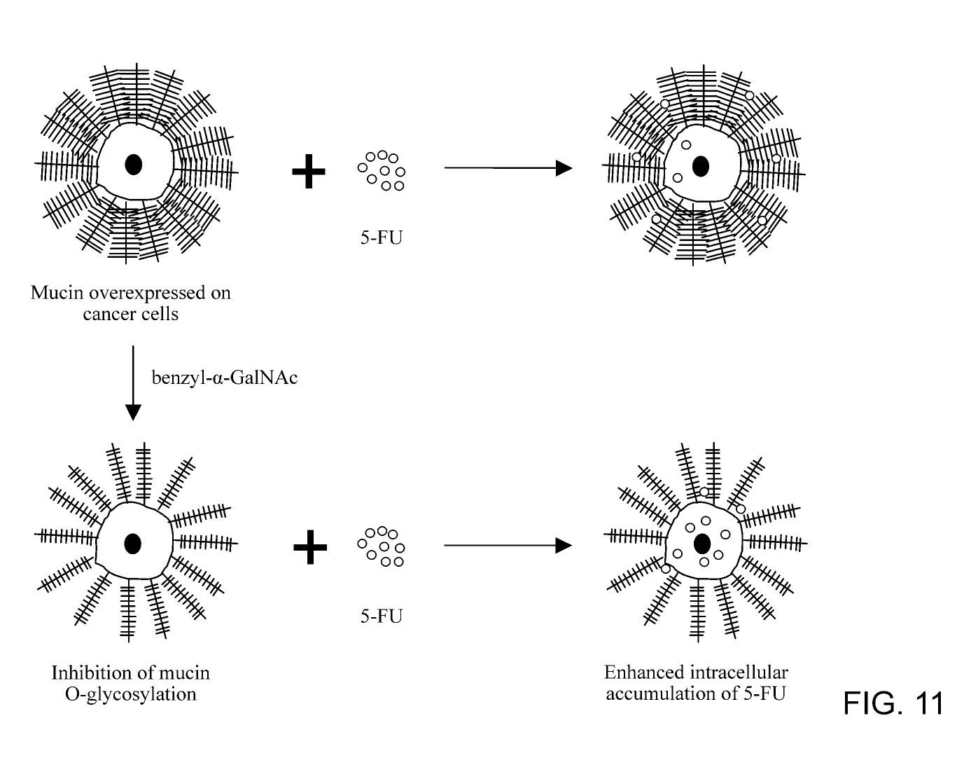

Pre-treatment of U-87 MG cells with benzyl-α-GalNAc did not alter the staining patterns of 5-FU following 1 hr or 4 hr of drug exposure (FIG. 10A and FIG. 10B).

Table 2 shows the fluorescence intensities corresponding to the levels of 5-FU in Capan-1 and U-87 MG cells following 1 hr and 4 hr of drug exposure.

TABLE 2
Intracellular Uptake of 5-Fluorouracil

| 5-FU exposure time | | 1 h | | 4 h |
|---|---|---|---|---|
| Benzyl-α-GalNAc | − | + | − | + |
| Capan-1 | 1696.9 ± | 7271.4 ± | 4253.0 ± | 3933.3 ± |
| | 693.7* | 3556.2* | 3045.9* | 2080.5* |
| U-87 MG | 3757.0 ± | 2346.9 ± | 3704.9 ± | 2989.4 ± |
| | 2457.1* | 1241.1 | 2262.9* | 1024.3* |

*Fluorescence intensities, arbitrary units
*P = 0.05

The fluorescence intensities observed in Capan-1 cells following 1 hr of 5-FU exposure were significantly (p=0.05) higher when cells were pre-treated with benzyl-α-GalNAc compared to 5-FU alone, whereas the fluorescence intensities for U-87 MG cells were similar in the presence and absence of the O-glycosylation inhibitor. Furthermore, the fluorescence intensities observed for both cell lines following 4 hr of 5-FU exposure were similar in the presence or absence of benzyl-α-GalNAc. These results suggest that the uptake of 5-FU by pancreatic cancer cells was limited by the overexpression of mucins on the cell surface. The most significant uptake of 5-FU by Capan-1 cells exposed to benzyl-α-GalNAc was observed following 1 hr of drug exposure. The fluorescence intensities observed following 4 hr of drug exposure were not significantly higher when compared to 1 hr of drug exposure. Several reports suggest that the conversion of 5-FU to its active metabolites is rapid. For example, in Ls174T colon cancer cells 5-FU metabolites were observed within 4 hr of 5-FU exposure. This would explain why there was no significant increase in 5-FU uptake following 4 hr of drug exposure when compared to 1 hr, as some of the drug may have been converted to its metabolites, but the antibody has no cross-reactivity to active metabolites of the prodrug. There were no alterations in intracellular accumulation of the drug in non-mucin expressing cells following exposure to benzyl-α-GalNAc, further supporting the functional role of mucins in cytotoxic drug therapy.

### Example 10

### Role of Type O-Glycosylation on the Cytotoxic Activity of Chemotherapeutic Agents of Various Molecular Weights and Sizes

This experiment examined the drug-barrier effect of mucin using chemotherapeutic agents of varying physico-chemical properties and mechanisms of action with pancreatic adenocarcinoma as an in vitro model.

Human pancreatic cancer cell morphology was visualized using light microscopy. Six hydrophilic chemotherapeutic agents namely cisplatin, gemcitabine, carboplatin, methotrexate, doxorubicin, vinblastine and six lipophilic agents namely lomustine, busulfan, camptothecin, etoposide, teniposide and paclitaxel with molecular weights ranging from approximately 200 to 900 were used. Two different concentrations of each drug type were evaluated in the presence and absence of heavily O-glycosylated chains with Benzyl-α-GalNAc as O-glycosylation inhibitor. Cell viability was determined using Sulforhodamine B assay. The percent change in cell viability was used to assess the overall benefit of reducing mucin in Capan-1 cells when different cytotoxic drugs are employed.

Cells were seeded at 1×10⁴ per ml in a 48-well plate. Following a 24 hr incubation period at 37° C. one row of cells was exposed to a predetermined concentration of benzyl-α-GalNAc solution prepared in growth medium, and another without the glycosylation inhibitor. Following 48 additional hr of incubation each row was washed with 1×PBS and treated with one of the chemotherapeutic agents prepared in cell growth medium. The growth inhibitory effect of the chemotherapeutic agent after 24 hr of exposure was determined by sulforhodamine B assay and percent of viable cells was calculated as follows:

$$Percent of cell viability = \frac{\begin{matrix}Fluorescence intensity of cells \\ treated with chemo agent\end{matrix} }{\begin{matrix}Fluorescence intensity of \\ non \text{-} treated cells ( control )\end{matrix} } \times 100$$

All drugs showed a significant decrease in cell viability when pretreated with benzyl-α-GalNAc. Percent increase in chemotherapeutic activity varied from 6% to 34%. The highest chemotherapeutic benefit of 33% was observed for Carboplatin (Mol.Wt. 371) and 34% for Camptothecin (Mol.Wt. 348).

Inhibition of mucin O-glycosylation enhanced drug uptake and cell kill in pancreatic adenocarcinoma cells. This trend appears to be size and molecular weight dependent. No significant difference in percent advantage was observed between two concentrations of any one drug.

### Example 11

### Method of Treating Lung Cancer

A patient presenting with lung cancer is administered guaifenesin (MUCINEX®) prior to treatment with a therapeutic agent that is typically used in treating lung cancer (e.g., a chemotherapeutic agent).

MUCINEX® is administered by mouth, usually every 12 hr with a full glass of water or as directed by the doctor. An adult patient should not take more than 4 MUCINEX® tablets in 24 hr. Children aged 6 to 12 years should not take more than 2 tablets in 24 hr, while children aged 2 to 6 years should not take more than 1 tablet in 24 hr.

MUCINEX® is administered anywhere from about 0.5 hr to about 96 hr before treatment with the therapeutic agent. The therapeutic agent is selected from etoposide, paclitaxel, camptothecin, lomustine, teniposide, fluoropyrimidines (e.g., 5-FU, FUrd, FUMP, and FdUMP), and combinations thereof. MUCINEX® may also be administered anywhere from about 0.1 hr to about 96 hr after treatment with the therapeutic agent.

It is expected that lung cancer patients will respond better to such treatment than if they had not been administered MUCINEX® prior to the administration of the therapeutic agent.

1. A method of treating a subject with a mucinous carcinoma, comprising administering to the subject a mucin inhibitor and/or a mucolytic agent and a therapeutic agent.

2. The method of claim 1, wherein the mucinous carcinoma is selected from the group consisting of pancreatic cancer, lung cancer, colon cancer, breast cancer, thyroid cancer, prostate cancer, and colorectal cancer.

3. The method of claim 1, wherein the mucin inhibitor or the mucolytic agent is administered prior to the administration of the therapeutic agent.

4. The method of claim 3, wherein the mucin inhibitor or the mucolytic agent is administered between about 0.5 hr to about 96 hr before the administration of the therapeutic agent.

5. The method of claim 1, wherein the mucin inhibitor or the mucolytic agent is administered at substantially the same time as the administration of the therapeutic agent.

6. The method of claim 1, wherein the therapeutic agent is selected from the group consisting of a chemotherapeutic agent, a radiomimetic, and a combination thereof.

7. The method of claim 1, further comprising administering at least one of a multidrug transporter inhibitor, an N-glycosylation inhibitor, and a sialyltransferase inhibitor with the mucin inhibitor and/or mucolytic agent.

8. A method of improving chemotherapeutic treatment in a subject in need thereof, comprising administering to the subject a mucin inhibitor and mucolytic agent prior to the administration of a chemotherapeutic agent, wherein the subject has improved response to the treatment compared with a patient not administered the mucin inhibitor or the mucolytic agent prior to the administration of the chemotherapeutic agent.

9. The method of claim 8, wherein the mucin inhibitor or the mucolytic agent is administered between about 0.5 hr to about 96 hr before the administration of the chemotherapeutic agent.

10. The method of claim 8, further comprising administering at least one of a multidrug transporter inhibitor, an N-glycosylation inhibitor, and a sialyltransferase inhibitor with the mucin inhibitor and/or mucolytic agent.

11. A method for improving the uptake of a therapeutic agent by a cell expressing or secreting higher levels of mucin than a normal cell of the same cell type, comprising contacting the cell with a mucin inhibitor and/or a mucolytic agent prior to contacting the cell with the therapeutic agent, wherein the therapeutic agent is taken up by the cell at a higher level than when the cell is not contacted with the mucin inhibitor or the mucolytic agent prior to contacting the cell with the therapeutic agent.

12. The method of claim 11, wherein the mucin inhibitor or the mucolytic agent is administered between about 0.5 hr to about 96 hr before contacting the cell with the therapeutic agent.

13. The method of claim 11, further comprising contacting the cell with at least one of a multidrug transporter inhibitor, an N-glycosylation inhibitor, and a sialyltransferase inhibitor with the mucin inhibitor and/or mucolytic agent.

14. A method for improving the ability of a ligand to bind to its cognate receptor on a cell surface, wherein the cell expresses or secretes higher levels of mucin than a normal cell of the same cell type, comprising contacting the cell with a mucin inhibitor and/or a mucolytic agent prior to contacting the cell with the ligand, wherein the ligand shows improved binding to its cognate receptor on the cell surface than when the cell has not been contacted with the mucin inhibitor or the mucolytic agent prior to contacting the cell with the ligand.

15. The method of claim 14, wherein the mucin inhibitor or the mucolytic agent is administered between about 0.5 hr to about 96 hr before contacting the cell with the ligand.

16. The method of claim 14, further comprising contacting the cell with at least one of a multidrug transporter inhibitor, an N-glycosylation inhibitor, and a sialyltransferase inhibitor with the mucin inhibitor and/or mucolytic agent.

17. A method for improving the ability of an antibody to bind to its antigen on a cell surface, wherein the cell expresses or secretes higher levels of mucin than a normal cell of the same cell type, comprising contacting the cell with a mucin inhibitor and/or a mucolytic agent prior to contacting the cell with the antibody, wherein the antibody shows improved binding to its antigen on the cell surface than when the cell has not been contacted with the mucin inhibitor or the mucolytic agent prior to contacting the cell with the antibody.

18. The method of claim 17, wherein the mucin inhibitor or the mucolytic agent is administered between about 0.5 hr to about 96 hr before contacting the cell with the antibody.

19. The method of claim 17, further comprising contacting the cell with at least one of a multidrug transporter inhibitor, an N-glycosylation inhibitor, and a sialyltransferase inhibitor with the mucin inhibitor and/or mucolytic agent.

20. A composition comprising a mucin inhibitor and/or a mucolytic agent and at least one of a multidrug transporter inhibitor, an N-glycosylation inhibitor, and a sialyltransferase.
